(19) United States
(12) Patent Application Publication
(10) Pub. No.: US 2018/0003721 A1
(43) Pub. Date: Jan. 4, 2018

(54) Metabolite Biomarkers Predictive Of Renal Disease In Diabetic Patients

(71) Applicant: Joslin Diabetes Center, Boston, MA (US)

(72) Inventors: Monika A. Niewczas, Boston, MA (US); Andrzej S. Krolewski, Needham, MA (US)

(73) Assignee: Joslin Diabetes Center, Boston, MA (US)

(21) Appl. No.: 15/539,022

(22) PCT Filed: Jan. 15, 2016
(86) PCT No.: PCT/US16/13661

§ 371 Date: Jun. 22, 2017

Related U.S. Application Data
(60) Provisional application No. 62/103,709, filed on Jan. 15, 2015.

(51) Int. Cl. G01N 33/68

(52) U.S. Cl. CPC G01N 33/6893; G01N 2800/7066; G01N 2800/50; G01N 2560/00; G01N 2800/042; G01N 2800/347

(57) ABSTRACT

The present invention relates to biomarkers that are predictive of renal disease in patients who have diabetes. The present invention also provides methods of using such biomarkers to predict the risk that a diabetic patient will develop renal disease, and/or to identify a patient who has diabetes as being in need of a therapy to prevent or delay the onset of a renal disease.

### RELATED APPLICATION

This application claims the benefit of U.S. Provisional Application No. 62/103,709, filed on Jan. 15, 2015. The entire teachings of the above application are incorporated herein by reference.

### GOVERNMENT SUPPORT

This invention was made with government support under Grant Nos. P30DK036836, DK41526, and DK67638 from National Institutes of Health (NIH). The government has certain rights in the invention.

### BACKGROUND OF THE INVENTION

Patients with End-Stage Renal Disease (ESRD) require dialysis or a kidney transplant for survival. Surprisingly, the incidence of ESRD due to diabetes has increased over the last 20 years despite improving hyperglycemia control and increased renoprotective drug use. To counteract this trend, it would be helpful to identify diabetic patients who are at risk for developing ESRD before they exhibit symptoms of renal disease.

Accordingly, there is an urgent need for biomarkers that are predictive of ESRD in diabetic patients.

### SUMMARY OF THE INVENTION

The present invention is based, in part, on the discovery of biomarkers that are predictive of renal disease in patients who have diabetes. Accordingly, in certain embodiments, the invention described herein relates to a method of predicting risk of developing renal disease in a patient who has diabetes. The method of predicting risk comprises the steps of a) determining the levels of at least three metabolites selected from the group consisting of pseudouridine, C-glycosyltryptophan, myoinositol, threitol, p-cresol sulfate, 2-hydroxyisovalerate, 2-hydroxyisocaproate, glutaryl carnitine, N2, N2-dimethylguanosine, phenylacetylglutamine, arabitol, gulono-1,4-lactone, erythritol, erythronate, N4-acetylcytidine, urate, 2-hydroxyisocaproate, 2-oxoisoleucine, and 2-oxoisocaproate in a sample (e.g., a serum sample, a plasma sample) taken from the patient; b) comparing the levels of the metabolites in the sample from the patient to control levels of the metabolites; and c) predicting that the patient is at risk for developing renal disease when the levels of the metabolites in the sample from the patient are significantly higher than the control levels of the metabolites.

In other embodiments, the invention relates to a method of identifying a patient who has diabetes as being in need of a therapy to prevent or delay the onset of a renal disease. The method of identifying a patient comprises the steps of a) determining the levels of at least three metabolites selected from the group consisting of pseudouridine, C-glycosyltryptophan, myoinositol, threitol, p-cresol sulfate, 2-hydroxyisovalerate, 2-hydroxyisocaproate, glutaryl carnitine, N2, N2-dimethylguanosine, phenylacetylglutamine, arabitol, gulono-1,4-lactone, erythritol, erythronate, N4-acetylcytidine, urate, 2-hydroxyisocaproate, 2-oxoisoleucine, and 2-oxoisocaproate in a sample (e.g., serum sample, plasma sample) taken from the patient; b) comparing the levels of the metabolites in the sample from the patient to control levels of the metabolites; and c) implementing a therapy to prevent or delay the onset of a renal disease in the patient when the levels of the metabolites in the sample from the patient are significantly higher than the control levels of the metabolites.

The methods described herein are useful for identifying diabetic patients who are at an increased risk for developing renal disease as early as 5-10 years prior to the occurrence of clinical symptoms of the disease.

### BRIEF DESCRIPTION OF THE DRAWINGS

FIG. 1. Stability of the common metabolites within individuals with type 2 diabetes in plasma samples taken 1-2 years apart. Spearman's rank correlation coefficients (r) are presented per individual measurements. The line and number represent median value per specific class.

FIGS. 2A and 2B. Multivariate analysis (volcano plot) of all common metabolites measured on the Metabolon platform and their association with progression to end-stage renal disease (ESRD) are demonstrated as a fold difference (x-axis) and significance adjusted for multiple comparisons and presented as q-values (y-axis). Uremic solutes comprise metabolites of interest in FIG. 2A and amino acids are metabolites of interest in FIG. 2B. Uremic solutes are not displayed in FIG. 2B. Common and stable metabolites of interest are represented as diamonds, common metabolites that are not stable over time are represented as empty circles, and all other common metabolites are represented as filled circles. Certain essential amino acids are indicated by name.

FIG. 3. Logistic regression analysis of the effect of the plasma concentration of metabolites identified as uremic solutes on the risk of progression to end-stage renal disease (ESRD) in patients with type 2 diabetes (T2D). Data are odds ratios and 95% confidence intervals (OR, 95% CI) estimated for an effect of 1 s.d. change of the metabolite. AER, albumin excretion rate; eGFR, estimated glomerular filtration rate; HbA1c, hemoglobin A1c.

FIG. 4. Logistic regression analysis of the effect of the plasma concentration of proteogenic amino acids and amino-acid derivatives on the risk of progression to end-stage renal disease (ESRD) in subjects with type 2 diabetes (T2D). Data are odds ratios and 95% confidence intervals (OR, 95% CI) estimated for an effect of 1 s.d. change of the metabolite. *Metabolite was not stable over time but is shown for its biological relevance. AER, albumin excretion rate; eGFR, estimated glomerular filtration rate; HbA1c, hemoglobin A1c.

FIG. 5. Hierarchical cluster analysis (Ward's method) of the metabolites significantly associated with progression to end-stage renal disease (ESRD). Separate clusters are delineated with broken lines. Distance scale is shown. C1-C6 represent respective clusters.

FIGS. 6A-6D. Association of the metabolites of the major biochemical classes: carbohydrates (FIG. 6A), lipids (FIG. 6B), nucleotides (FIG. 6C) and other metabolites (FIG. 6D) with progression to ESRD in subjects with T2D in the multivariate analysis of the global metabolomics profiling. Data are presented as the volcano plot for the well detectable metabolites measured in plasma stratified by their performance. The X-axis represents the fold difference in logarithmic (base 2) scale and the Y-axis represents significance, p-value adjusted by multiple comparisons (q value) in negative logarithmic (base 10) scale. Uremic solutes and amino acids are not displayed.

### DETAILED DESCRIPTION OF THE INVENTION

The incidence of End-Stage Renal Disease (ESRD) due to type 2 diabetes (T2D) increased over the last 20 years despite improving hyperglycemia control and increased renoprotective drugs use. (1) Clearly, a better understanding of the determinants responsible for progression to ESRD in T2D is urgently needed if this “epidemic” is to be contained.

Recently developed platforms for global metabolomic profiling are capable of examining hundreds of metabolites, so they are excellent tools to study complex metabolic alterations associated with progression of diabetic nephropathy.(2, 3) Reliable metabolomic data can be obtained with liquid or gas chromatography coupled with mass spectrometry (LC/GC-MS) or NMR spectroscopy. Among those, MS-based platforms are the most sensitive.(2, 4-6)

One of the hallmarks of progression to ESRD is plasma accumulation of certain metabolites, the so-called uremic solutes.(7-10) However, it is becoming apparent that increase in the levels of uremic solutes in blood may be more than a simple reflection of impaired kidney function.(11-13) The kidney is a key organ involved in the handling of major biochemical classes of metabolites. Kidney function includes filtration of metabolites via glomeruli, followed by their tubular secretion/reabsorption and synthesis/degradation in various components of the renal parenchyma. At present it is unclear whether elevated levels of uremic solutes precede or follow renal impairment. For example, elevated plasma concentration of uremic solutes may contribute to glomerular as well as tubular damage in diabetic nephropathy, and damage to those two components have been demonstrated in early nephropathy.(14, 15) Various alterations of certain biochemical classes of metabolites (amino acids, in particular) have been also reported in the associations with insulin resistance, type 2 diabetes or chronic kidney injury per se.(16-19)

To date, few metabolomic studies focusing on diabetic nephropathy have been performed in experimental models (20, 21) or in humans.(22-25) Nevertheless, the comparisons were either cross-sectional or focused on albuminuria progression rather than on the kidney failure, the ultimate outcome of the diabetic nephropathy.(22-25)

A description of example embodiments of the invention follows.

In certain embodiments, the present invention relates to a method of predicting risk of developing renal disease in a patient who has diabetes, comprising the steps of a) determining the levels of at least four metabolites selected from the group consisting of pseudouridine, C-glycosyltryptophan, myoinositol, threitol, p-cresol sulfate, 2-hydroxyisovalerate, 2-hydroxyisocaproate, glutaryl carnitine and N2, N2-dimethylguanosine in a sample taken from the patient; b) comparing the levels of the metabolites in the sample from the patient to control levels of the metabolites; and c) predicting that the patient is at risk for developing renal disease when the levels of the metabolites in the sample from the patient are significantly higher than the control levels of the metabolites.

As used herein, “patient” refers to a mammal (e.g., human, horse, cow, dog, cat). Preferably, the patient is a human.

In some embodiments, the patient has type 2 diabetes. In other embodiments, the patient has type 1 diabetes.

The methods disclosed herein are useful for predicting the risk of developing renal disease in diabetic patients before the onset of symptoms of a renal disease. Accordingly, in some embodiments, the patient does not have symptoms of a renal disease. In a particular embodiment, the patient has normal renal function. In another embodiment, the patient has mildly impaired renal function.

In one embodiment, the methods disclosed herein are useful for predicting the risk of diabetic nephropathy in a patient who has diabetes. In a further embodiment, the methods disclosed herein are useful for predicting the risk of ESRD in a diabetic patient.

The methods disclosed herein comprise the step of determining the levels of at least three metabolites selected from the group consisting of pseudouridine, C-glycosyltryptophan, myoinositol, threitol, p-cresol sulfate, 2-hydroxyisovalerate, 2-hydroxyisocaproate, glutaryl carnitine, N2, N2-dimethylguanosine, phenylacetylglutamine, arabitol, gulono-1,4-lactone, erythritol, erythronate, N4-acetylcytidine, urate, 2-hydroxyisocaproate, 2-oxoisoleucine, and 2-oxoisocaproate in a sample taken from the patient.

The sample that is taken from the patient can be any suitable bodily fluid sample (e.g., blood, plasma, serum, spinal fluid, lymph fluid, urine, amniotic fluid). In a particular embodiment, the sample is a plasma sample. In another embodiment, the sample is a serum sample.

The term “at least three metabolites” encompasses any combination of three or more (e.g., four, five, six, seven, eight, nine, ten, eleven, twelve, thirteen, fourteen, fifteen, sixteen, seventeen, eighteen or nineteen) metabolites selected from the group consisting of pseudouridine, C-glycosyltryptophan, myoinositol, threitol, p-cresol sulfate, 2-hydroxyisovalerate, 2-hydroxyisocaproate, glutaryl carnitine, N2, N2-dimethylguanosine, phenylacetylglutamine, arabitol, gulono-1,4-lactone, erythritol, erythronate, N4-acetylcytidine, urate, 2-hydroxyisocaproate, 2-oxoisoleucine, and 2-oxoisocaproate. Thus, any combination of three or more of these metabolites can be used to predict the risk of developing a renal disease. Preferably, the “at least three metabolites” include pseudouridine and/or C-glycosyltryptophan. More preferably, the “at least three metabolites” includes a nucleotide derivative (e.g., pseudouridine, N2, N2-dimethylguanosine), an amino acid derivative (e.g., C-glycosyltryptophan), a polyol (e.g., myoinositol, threitol), a phenyl compound (e.g., p-cresol sulfate), a branched amino acid derivative (e.g., 2-hydroxyisovalerate, 2-hydroxyisocaproate) and a branched chain acylcarnitine (e.g., 2-hydroxyisocaproate, glutaryl carnitin). In a particular embodiment, the levels of all metabolites in the group consisting of pseudouridine, C-glycosyltryptophan, myoinositol, threitol, p-cresol sulfate, 2-hydroxyisovalerate, 2-hydroxyisocaproate, glutaryl carnitine and N2, N2-dimethylguanosine are determined.

Suitable techniques and reagents for detecting levels of metabolites in a sample from a patient are known in the art. For example, levels of metabolites in a sample can be determined using mass spectrometry (MS) or NMR spectroscopy.

In a particular embodiment, levels of metabolites in a sample are determined using mass spectrometry (e.g., ESI MS, MALDI-TOF MS, tandem MS (MS/MS)). In general, mass spectrometry involves ionizing a sample containing one or more molecules of interest, and then m/z separating and detecting the resultant ions (or product ions derived therefrom) in a mass analyzer, such as (without limitation) a quadrupole mass filter, quadrupole ion trap, time-of-flight analyzer, FT/ICR analyzer or Orbitrap, to generate a mass spectrum representing the abundances of detected ions at different values of m/z. See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., “Protein chip surface enhanced laser desorption/ionization (SELDI) mass spectrometry: a novel protein biochip technology for detection of prostate cancer biomarkers in complex protein mixtures,” Prostate Cancer and Prostatic Diseases 2: 264-76 (1999); and Merchant and Weinberger, “Recent advancements in surface-enhanced laser desorption/ionization-time of flight-mass spectrometry,” Electrophoresis 21: 1164-67 (2000), each of which is hereby incorporated by reference in its entirety, including all tables, figures, and claims.

Levels of metabolites can also be determined using a Metabolon (Durham, N.C.) MS platform, as described herein.

In some embodiments, the sample from the patient is subjected to a liquid chromatography (LC) or gas chromatography (GC) purification step prior to mass spectrometry (LC/GC-MS). Methods of coupling liquid chromatography techniques to MS analysis are known in the art.

The methods disclosed herein further comprise the steps of comparing the levels of the metabolites in the sample from the patient to control levels of the metabolites and predicting that the patient is at risk for developing renal disease when the levels of the metabolites in the sample from the patient are significantly higher than the control levels of the metabolites.

A control level for a given metabolite can be obtained, for example, from a sample, or collection of samples, taken from diabetic patients who did not develop renal disease. Alternatively, a control level for a given metabolite can be based on a suitable reference standard. The reference standard can be a typical, normal or normalized range of levels, or a particular level, of a metabolite. The standards can comprise, for example, a zero metabolite level, the level of a metabolite in a standard cell line, or the average level of a metabolite previously obtained for a population of normal human controls. Thus, the methods disclosed herein do not require that the level of a metabolite be assessed in, or compared to, a control sample.

In accordance with the invention, a patient is predicted to be at risk for developing renal disease when the levels of the metabolites in the sample from the patient are significantly higher than the control levels of the metabolites. A statistically significant difference (e.g., an increase, a decrease) in the level of a metabolite between two samples, or between a sample and a reference standard, can be determined using an appropriate statistical test(s), several of which are known to those of skill in the art. In a particular embodiment, a t-test (e.g., a one-sample t-test, a two-sample t-test) is employed to determine whether a difference in the level of a metabolite is statistically significant. For example, a statistically significant difference in the level of a metabolite between two samples can be determined using a two-sample t-test (e.g., a two-sample Welch's t-test). A statistically significant difference in the level of expression of a gene between a sample and a reference standard can be determined using a one-sample t-test. Other useful statistical analyses for assessing differences in gene expression include a Chi-square test, Fisher's exact test, and log-rank and Wilcoxon tests.

As used herein, patient who is “at-risk” for developing renal disease has a level of risk for developing a renal disease that is higher than the level of risk for an individual represented by the relevant baseline population.

In other embodiments, the invention relates to a method of identifying a patient who has diabetes as being in need of a therapy to prevent or delay the onset of a renal disease, comprising the steps of a) determining the levels of at least four metabolites selected from the group consisting of pseudouridine, C-glycosyltryptophan, myoinositol, threitol, p-cresol sulfate, 2-hydroxyisovalerate, 2-hydroxyisocaproate, glutaryl carnitine and N2, N2-dimethylguanosine in a sample taken from the patient; b) comparing the levels of the metabolites in the sample from the patient to control levels of the metabolites; and c) implementing a therapy to prevent or delay the onset of a renal disease in the patient when the levels of the metabolites in the sample from the patient are significantly higher than the control levels of the metabolites.

As defined herein, “therapy” is the administration of a particular therapeutic or prophylactic agent to a subject (e.g., a non-human mammal, a human), which results in a desired therapeutic or prophylactic benefit to the subject (e.g., prevention or delay in the onset of a renal disease).

A suitable therapy for preventing or delaying the onset of a renal disease in a patient can be readily determined by a skilled medical professional (e.g., a physician, such as a nephrologist), taking into account various factors including, but not limited to, the patient's age, weight, medical history, and sensitivity to drugs. Exemplary therapies for preventing or delaying the onset of a renal disease include, for example, administration of drugs to treat hypertension, dietary changes, exercise, weight loss, glycemic control, proteinuria therapies, and albuminuria therapies, among others at least three.

Preferably, the therapy is implemented early enough to prevent or delay the onset of renal disease in the patient. In some embodiments, the therapy is implemented before the patient shows any clinical symptoms of renal disease. According to the invention, the methods disclosed herein can identify diabetic patients who are at risk for developing renal disease about 5-10 years, or more, before the occurrence of clinical symptoms of renal disease.

### Example: Uremic Solutes and Risk of End-Stage Renal Disease in Type 2 Diabetes: Metabolomic Study

Study Groups and Methods:

Study Group

Between 1991 and 1995, a cohort with T2D was recruited into the Joslin Study of the Genetics of Type 2 Diabetes and Kidney Complications (half with normoalbuminuria and half with microalbuminuria or proteinuria). The cohort was followed-up until 2004 for the occurrence of ESRD or death unrelated to ESRD. Details of the recruitment, examination, and follow-up of this cohort were already published elsewhere.(26) Among 410 individuals, 59 developed ESRD and 84 died without ESRD as ascertained by United States Renal Data System(1), National Death Index(76) and medical records review.

For this nested case-control study, 40 out of 56 incident cases of ESRD who had sufficient stored plasma samples at baseline available were selected. A group of 40 subjects from among those who survived and were without ESRD as of the end of follow-up were selected as controls. They were grouped-matched with cases with regard to gender, age, and baseline eGFR. For comparison of cases and controls baseline clinical characteristics were used as reported previously.(26)

As a small reproducibility study, ten study subjects (balanced by caseness status) had plasma samples selected 2.2+0.8 years after baseline. All plasma samples were stored at −70° C. until analysis. The study protocol and informed consent procedures were approved by the Joslin Diabetes Center Institutional Review Board.

Global Metabolomics Profiling

All plasma samples (80 baseline and 10 from the early follow up timepoint) were subjected to global metabolomic profiling (Metabolon, Inc, Durham, N.C.). Sample extracts were prepared to recover a wide range of chemically diverse metabolites. The LC/MS portion of the platform incorporates a Waters Acquity UPLC system and a Thermo-Finnigan LTQ mass spectrometer, including an electrospray ionization (ESI) source and linear ion-trap (LIT) mass analyzer. The GC column is 5% phenyl dimethyl silicone and the temperature ramp is from 60° to 340° C. in a 17-minute period. All samples were then analyzed on a Thermo-Finnigan Trace DSQ fast-scanning single-quadrupole mass spectrometer using electron impact ionization. Biochemicals were subsequently identified by comparison to library entries of over 2400 purified standards for distribution to both the LC/GC-MS platforms for determination of their analytical characteristics.

After peak identification and quality control filtering (signal greater than 3 times background, retention index within a pre-specified platform-dependent window, ion quantification to library match within 0.4 m/z, and the MS/MS forward and reverse scores), the metabolites' relative concentrations were obtained from median-scaled day-block normalized data for each compound. Repeated samples from the same individual were run the same day. The metabolite was defined as common, if it was present in at least 80% of the individuals in the study group, and as stable over time, when Spearman correlation coefficient between two measurements taken from the same individual was ≧0.4. For detailed information regarding all metabolites detected by global profiling, please refer to Supplemental Table 1.

Uremic Solutes

The European Uremic Toxins (EUTox) Work Group, initiated in 1999, consists of 24 European Research Institutes and provides the most comprehensive encyclopedic list of systematically and critically reviewed uremic solutes/toxins.(7, 9) Metabolites measured with the global profiling were classified as uremic solutes/toxins based on the EUTox list prepared in 2003, revisited in 2012 as well as based on selected relevant other publications.(7-9, 11, 13, 32) Seventy eight uremic solutes are available in the Metabolon library. For detailed information of the detectable uremic solutes in this study, please see Supplemental Table 2.

Targeted Quantitative Measurements of Metabolites

Quantitative measurements of phenyl and indole compounds (p-cresol and indoxyl sulfate, phenylacetylglutamine and hippurate) were performed as a collaborative effort with Dr. T. W. Meyer's laboratory by stable isotope dilution LC/MS/MS using p-cresol-d₈-sulfate (synthesized from p-cresol d₈³¹ (Cambridge Isotopes)), indoxyl-2,4,5,6,7-d₅-sulfate (Isosciences), and N-benzoyl-d5-glycine and Nα-(phenyl-d5-acetyl)-L-glutamine (both C/D/N Isotopes) as internal standards. LC was performed on a Kinetex, C18 column maintained at 30° C. MS was performed on an Agilent 6430 Triple Quadrupole mass spectrometer with electrospray ionization in the negative mode (Agilent Technologies). Solute concentrations were calculated using manufacturer's software (MassHunter Quant). Ion transitions used for quantitation were m/z 187/107 for p-cresol sulfate, m/z 212/80 for indoxyl sulfate, m/z 178.1/134.2 for hippurate, and m/z 263.2/145.1 for phenylacetylglutamine with corresponding transitions for the deuterated internal standards. Spiked recoveries for all the metabolites were between 93 to 108%.

Quantitative measurements of modified nucleotide derivatives and amino acids were performed as a collaborative effort with Dr. Pennathur's laboratory. Isotopically labeled ¹³C₆ tyrosine and ¹³C₆ phenylalanine (Cambridge Isotope Laboratories, Andover, Mass., USA), ¹³C₁ ¹⁵N₂ uracil (Movarek Biochemicals and Radiochemicals Brea, Calif., USA) were used to quantify the respective authentic compounds. Authentic pseudouridine (Santa Cruz Biotechnology, Dallas, Tex., USA) and N2N2-dimethylguanosine (Biolog Life sciences Institute, Bremen, Germany) were utilized as standards to construct calibration curves. Plasma samples were subjected to protein precipitation with 1:1 v/v 100% acetonitrile after addition of isotopically labeled internal standards. The supernatant containing the metabolites was subjected to liquid chromatography-electrospray ionization tandem mass spectrometry (LC-ESI-MS/MS) and metabolites were quantified using multiple reaction monitoring (MRM) in the MS/MS positive ion acquisition mode. For LC/ESI/tandem MS experiments, we utilized an Agilent 6410 triple Quadrupole MS system equipped with an Agilent 1200 LC system and an ESI source. Luna C-18 column was used for the LC separation. The mobile phase is 0.1% Formic acid (Solvent A) and acetonitrile with 0.1% formic acid (Solvent B). The following transitions were monitored for each of the analyte: a) m/z 113 to m/z 70 and m/z 116 to m/z 71 for uracil and ¹³C₁¹⁵N₂ uracil; b). m/z 166/120 and m/z 172/126 for phenylalanine and ¹³C₆ phenylalanine; c) m/z 182/136 and m/z 188/142 for tyrosine and ¹³C₆ tyrosine, and d) m/z 245/209 and m/z 312/180 for pseudouridine and N2,N2-dimethylguanosine respectively. For analytes with available corresponding isotopic standard the quantification was computed by the ratio of the peak area of the analyte compared with the known amount of the isotopic standard. For pseudouridine and N2,N2-dimethylguanosine calibration curves were created with the authentic nucleoside spiked in the biological matrix. Extraction efficiency and quality control were monitored with the peak area of the labeled stable isotopes in control plasma samples spaced throughout the run to monitor intra and inter run efficiency. Amino acids. Quantification of amino acid levels were performed using the “EZ:faast” kit for free amino acid analysis from Phenomenex (Torrance, Calif., USA) with Norvaline added as an internal standard as described previously using an Agilent 6890 GC-MS system.(79) For the results of amino acids measured on two platforms, please see the Supplemental Table 3.

Myo inositol measurements were performed as a collaborative effort with Dr G. Berry's laboratory (Boston Childrens Hospital, Boston, Mass.). Myo-inositol was purchased from Sigma-Aldrich (USA). The internal standard (IS), [2H6]-myo-inositol, was purchased from Cambridge Isotope Laboratories, Inc. (MA, USA). The separation of samples and standards was performed with Shimadzu Prominence high performance liquid chromatography (HPLC) unit and a SUPELCOGEL Pb (300×7.8 mm; 5 μm; Supelco UK) column, with the column temperature set at 85° C. The HPLC was coupled to a 5500 QTRAP hybrid dual quadrupole ion trap mass spectrometer (AB Sciex, Ontario, Canada) operating in negative-ion mode. The MRM transitions monitored were 179/87 for myo-inositol and 185.1/89 for [2H6]-myo-inositol. Samples were run in duplicate and measurements with coefficient variation (CV)<10% were considered for analysis.

Urate was measured in the clinical laboratory of the Joslin Diabetes Center, Boston, Mass. using an enzymatic colorimetric assay kit and read on the Roche Modular P Chemistry analyzer (Roche Diagnostics).(80) The inter-assay CV range is 1.9% in this laboratory.

Data Analysis

Differences in clinical characteristics between the two study groups were tested by analysis of variance for continuous variables and chi-square test for categorical variables. Preliminary data cleaning included investigations of detectability, batch effects and outliers at the metabolite- and individual-levels (heatmap, principal component procedures; data not shown).(29, 77) Volcano plots were generated for common metabolites based on the fold difference between the outcome groups and the p-value obtained in a general linear model. Adjustment for multiple comparison was performed with a positive false discovery rate (pFDR) q value <0.05 for significance.(78) The effect of a metabolite was estimated using logistic regression. After transformation of the metabolite concentration to normally distributed ranks, its effect on risk of progression was expressed as the odds ratio for a one standard deviation difference.(26) Clinical covariates and metabolites measured quantitatively were transformed to their (base 10) logarithms for the logistic analysis. Non-common metabolites were analyzed as categorical variables by chi-square, but this analysis did not result in identifying additional significant metabolites (data not shown).

Correlations between the continuous variables were examined with Spearman rank correlation. Data reduction was carried out with hierarchical cluster analysis using the Ward method based on values transformed to normally distributed ranks. Top metabolites from the multivariate volcano plots analysis were included. More than a half of the detected metabolites lacked pathway identifiers, preventing us from a comprehensive canonical pathway analysis. Data analysis was performed with SAS 9.3 and JMP Pro 9.0.0 softwares (Cary, N.C.).

Results

Study Groups and their Characteristics:

A cohort with T2D patients attending the Joslin Clinic was recruited into the Joslin Study of the Genetics of Kidney Complications. Of the 509 patients examined between 1992 and 1996. 410 were followed until the end 2004. During 8-12 years of follow-up 59 (14.4%) patients developed ESRD, 84 (20%) died without progressing to ESRD and 267 (65.1%) remained alive without progressing to ESRD. Details of the follow-up study were already published.(26)

For the present nested case-control study, 40 patients who developed ESRD (cases of progressors to ESRD) were selected and matched them with 40 patients who were alive as of 2004 without ESRD (controls for non-progressors). Of the 80 patients, 75 identified themselves as Caucasians of European origin. Baseline characteristics of the two selected study groups are summarized in Table 1. The groups were very similar with regard to most clinical characteristics. Progressors, however, had higher urinary albumin excretion and slightly lower eGFR. Despite the differences noted in median AER and mean eGFR, there was substantial overlap of the distributions in the two study groups. At baseline the majority of progressors and non-progressors were in CKD stage 2. CKD stage 3 was present in 7% of controls and 22% of cases, respectively. Overall the distribution of CKD stages was not statistically different between the study groups. 87% of non-progressors had annual eGFR decrease less than 3.5 ml/min/1.73 m². The median (25^{th}, 75^{th} percentile) decrease was −1.95 (−3.2, −0.8) ml/min/1.73 m² and the slope was determined based on the serial creatinine measurements over 7.6 (6.5-12.4) years. The study groups did not also differ regarding baseline plasma levels of parathormone.

TABLE 1
Baseline characteristics of subjects with T2D selected for nested case-
control study

| | Study groups | | |
|---|---|---|---|
| | Non- | ESRD | |
| | progressors, | progressors, | |
| | controls | cases | |
| Baseline characteristic | (n = 40) | (n = 40) | P-value^{a} |
| Male (%) | 62 | 55 | NS |
| Age (years) | 56 ± 11 | 59 ± 7 | NS |
| Duration of diabetes (years) | 13 ± 7 | 17 ± 7 | NS |
| Body mass index (kg/m²) | 29.7 ± 5.9 | 31.5 ± 7.1 | NS |
| HbA1c (%) | 8.8 ± 1.4 | 9.3 ± 2.0 | NS |
| Serum cholesterol (mg/dl) | 241 ± 62 | 234 ± 54 | NS |
| Systolic blood pressure | 135 ± 25 | 142 ± 32 | NS |
| (mmHg) | | | |
| Antihypertensive/ | 74% | 62% | NS |
| renoprotective | | | |
| treatment (%) | | | |
| ACR (μg/g creatinine) | 308 (70, 471) | 957 (382, 2265) | <0.0001 |
| eGFR (ml/min per 1.73 m²) | 87 ± 23 | 75 ± 19 | <0.01 |
| CKD | | | |
| Stage 1 | 38% | 20% | |
| Stage 2 | 55% | 58% | NS |
| Stage 3 | 7% | 22% | |
| Parathormone (pg/ml) | 15 (9, 20) | 16 (10, 31) | NS |
| Length of the follow-up | 10 (8, 12) | 7 (4, 9)^{b} | 0.008 |
| (years) | | | |

Abbreviations:
ACR, albumin-to-creatinine ratio;
CKD, chronic kidney disease;
eGFR, estimated glomerular filtration rate;
ESRD, end-stage renal disease;
HbA1c, hemoglobin A1c;
T2D, type 2 diabetes.
Proportion, mean ± s.d., or median (25th, 75th percentile) are presented.
^{a}Bonferroni corrected.
^{b}Until ESRD development.

Results of Global Metabolomic Analysis:

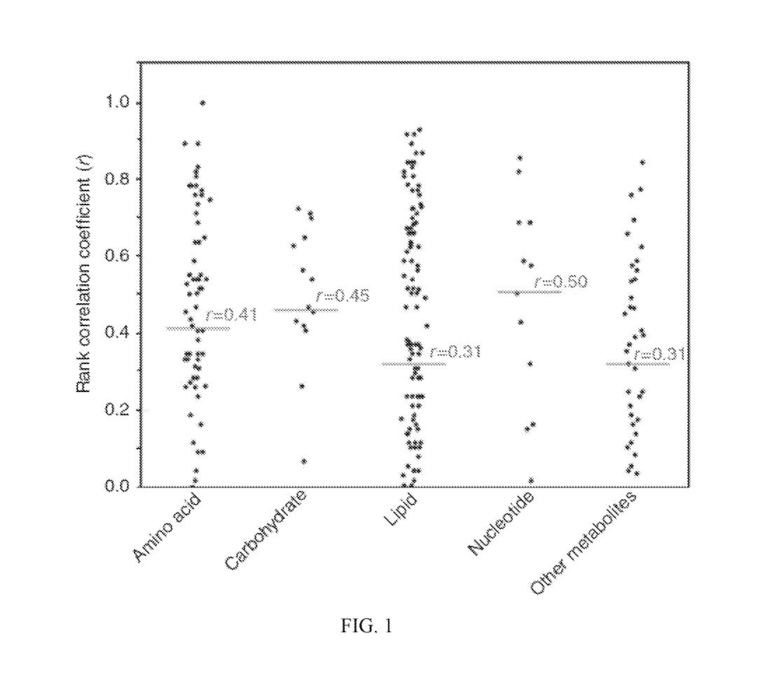

Baseline plasma samples from study subjects were run on the Metabolon platform against a library containing mass spectra for 2400 chemically identified metabolites. A total of 445 named metabolites were detected. Of these, 183 belonged to drug-related metabolites or had low detectability and were excluded from further analysis. The remaining 262 were detected in at least 80% of the study subjects and were designated as “common.” The assay was repeated in the follow-up plasma sample taken one to three years later for a subgroup of study patients to estimate intra-individual variation for each metabolite. The results by biochemical classes are summarized in FIG. 1 and presented in details in supplemental Table 1. A rank correlation coefficient ≧0.4 between these paired samples was used to distinguish metabolites with persistent or transient concentrations. By this criterion, 119 of the common metabolites (45%) tracked well over time within individual and thus were considered persistent, defined here as “stable” and of particular interest, which remains in accordance with the systematic report by Floegel et al. (27)

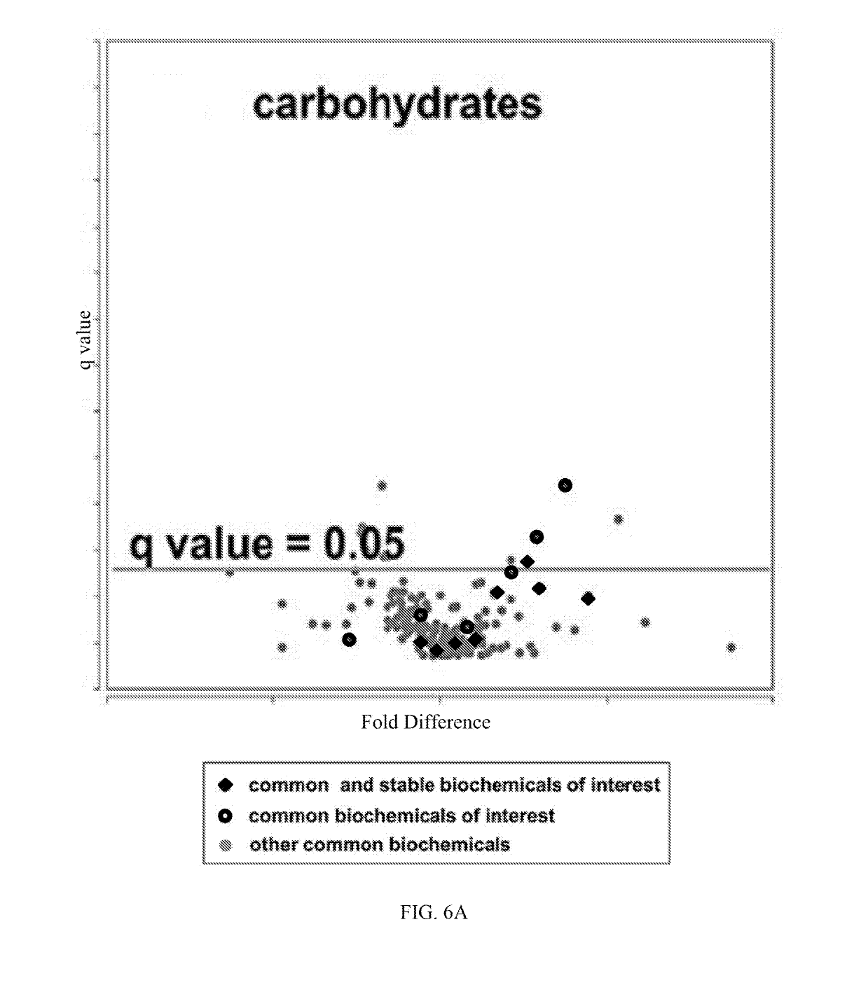

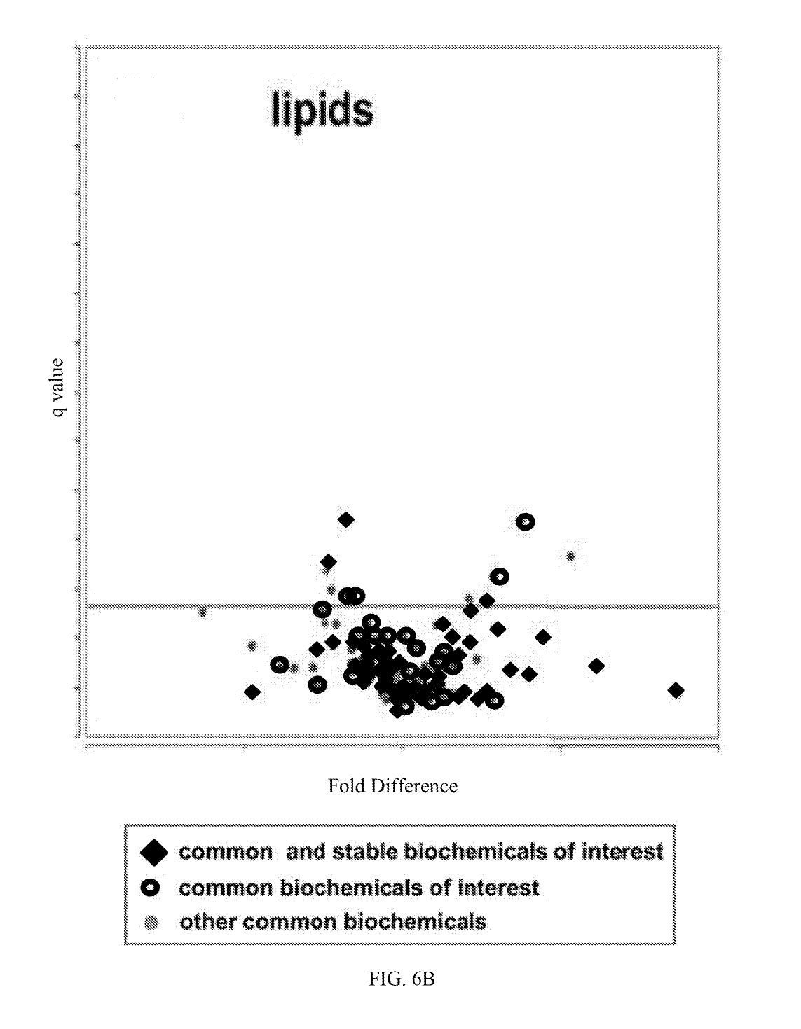

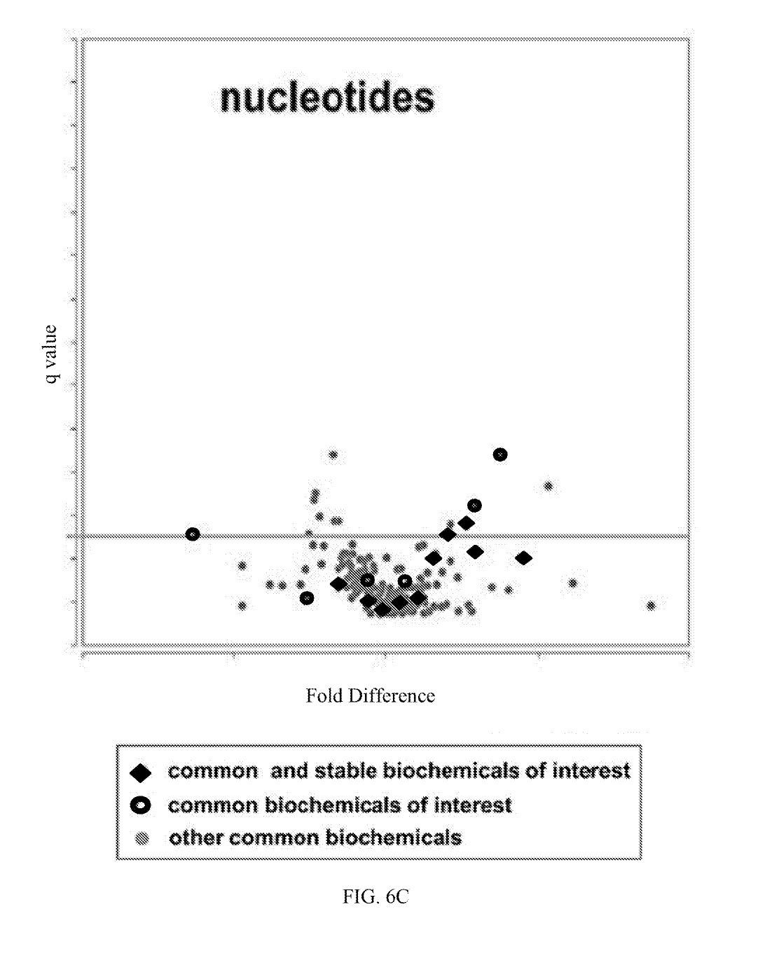

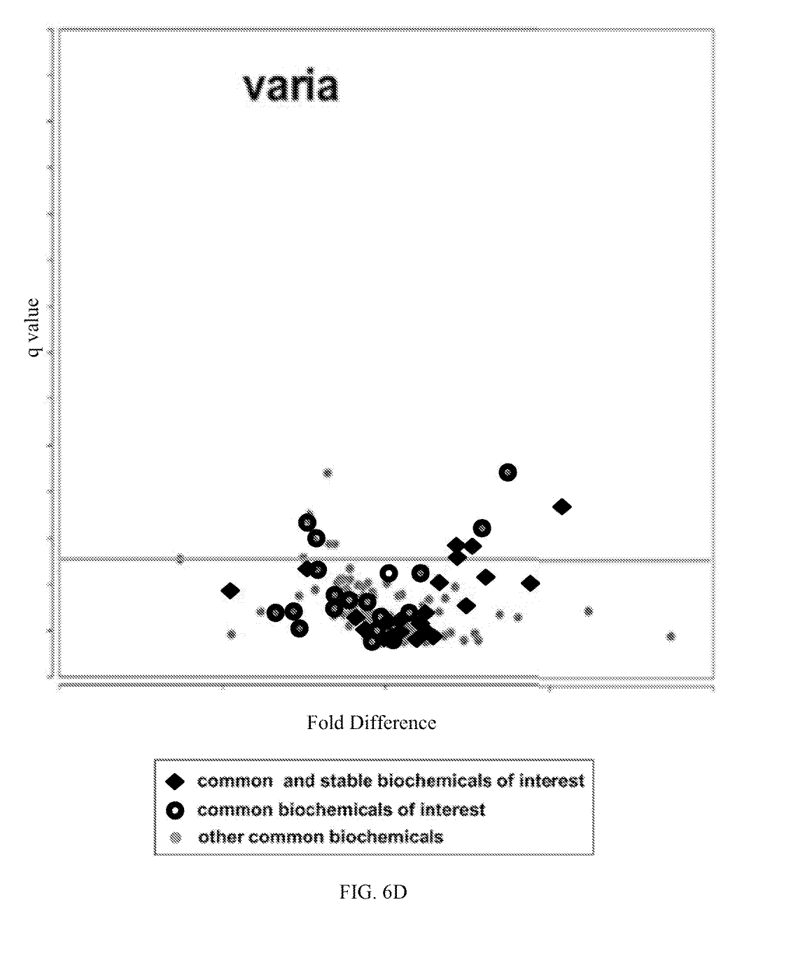

Fold differences between the mean plasma concentration in progressors to ESRD and non-progressors were analyzed for the 262 common metabolites (Table 2). In the analysis of fold difference, 49 (19%) were significant after adjusting for multiple comparisons (q value <0.05). Among the subset of 119 metabolites stable over time, 28 (23%) differed significantly from 1.0. These results are summarized in Table 2 according to biochemical class and whether the metabolite has been identified as a uremic solute. The proportion of metabolites associated with ESRD progression was similar regardless of whether the metabolite was stable over time. Nevertheless, further analysis focused on the stable ones because their involvement in a prolonged process such as progression to ESRD is more plausible. Among the biochemical classes, the proportion associated with ESRD was high for amino acids and their derivatives, carbohydrates, and modified nucleotides (40%, 42%, and 57%, respectively), intermediate (16%) for other metabolites, and low (4%) for lipids (see FIGS. 6A-6D).

TABLE 2
Summary of global metabolomic analysis: frequency of significant fold
differences between plasma concentrations in cases (who subsequently
progressed to ESRD) and controls (did not progress) according to type
of metabolite and its recognition as a uremic solute.

| | All common | | | |
|---|---|---|---|---|
| | metabolites | | Uremic solutes | |
| | | Associated | | Associated |
| | | with | | with |
| | Total, | ESRD^{a}, | Total, | ESRD^{a}, |
| Biochemical class | n | n (%) | n | n (%) |
| Lipids | | | | |
| Common metabolites^{b} | 126 | 4 (3%) | 0 | 0 (0%) |
| Stable metabolites^{c} | 51 | 2 (4%) | 0 | 0 (0%) |
| Amino acids and derivatives | | | | |
| Common metabolites^{b} | 67 | 25 (37%) | 10 | 3 (30%) |
| Stable metabolites^{c} | 35 | 14 (40%) | 6 | 2 (33%) |
| Carbohydrates | | | | |
| Common metabolites^{b} | 20 | 9 (45%) | 6 | 6 (100%) |
| Stable metabolites^{c} | 14 | 6 (42%) | 5 | 5 (100%) |
| Nucleotides | | | | |
| Common metabolites^{b} | 12 | 7 (58%) | 10 | 7 (70%) |
| Stable metabolites^{c} | 7 | 4 (57%) | 5 | 4 (80%) |
| Other metabolites | | | | |
| Common metabolites^{b} | 37 | 4 (11%) | 1 | 0 (0%) |
| Stable metabolites^{c} | 12 | 2 (16%) | 0 | 0 (0%) |
| Total | | | | |
| Common metabolites^{b} | 262 | 49 (19%) | 27 | 16 (55%) |
| Stable metabolites over time^{c} | 119 | 28 (23%) | 18 | 12 (67%) |

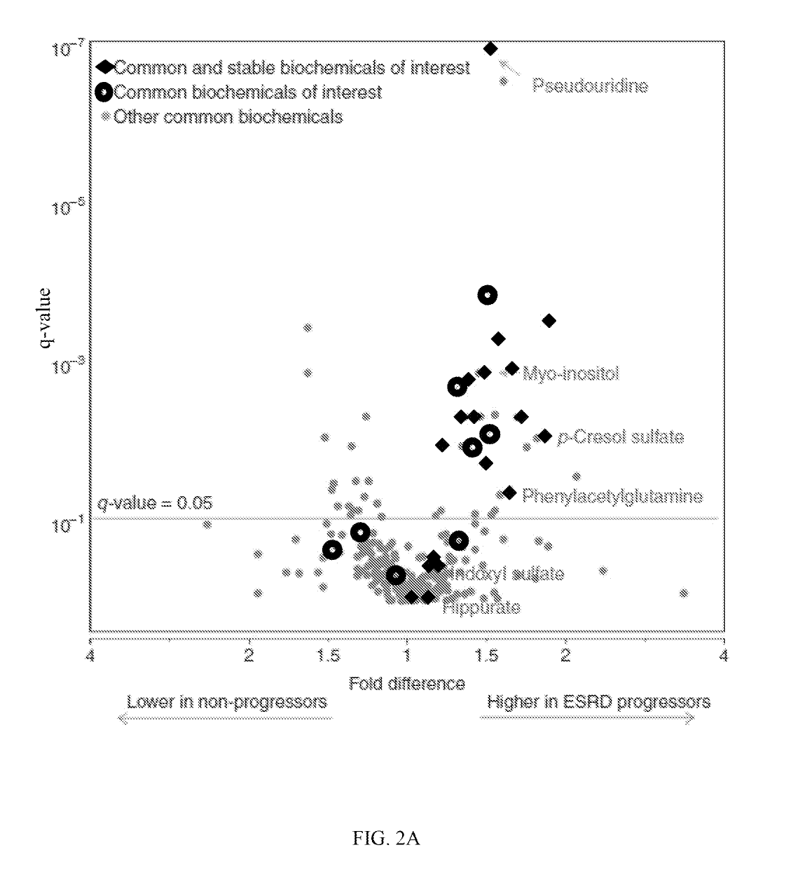

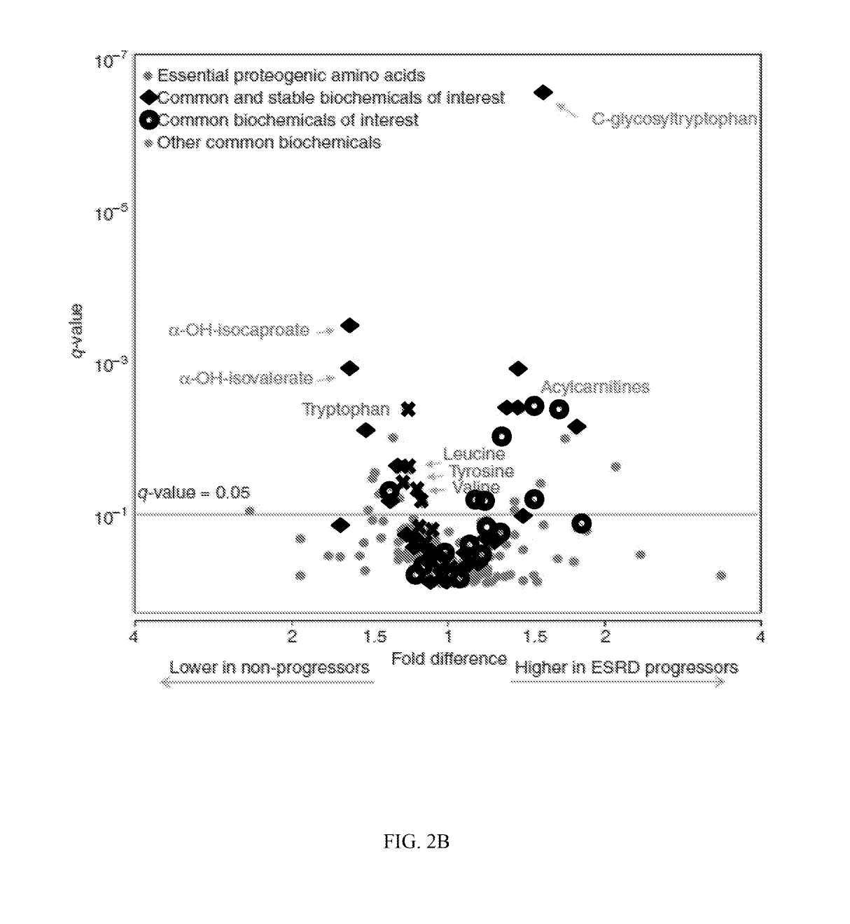

Abbreviation: ESRD, end-stage renal disease.
^{a}Values of fold difference were significantly different from 1.0 at a q-value o0.05. See also FIGS. 2a and b.
^{b}Detectable in plasma of X80% of patients.
^{c}Common and stable over time, that is, Spearman's correlation coefficient X0.4 between measurements taken 1-2 years apart from the same individual. Out of 29 stable metabolites associated with ESRD, five were not examined further.

Progression to ESRD According to Plasma Concentration of Uremic Solutes:

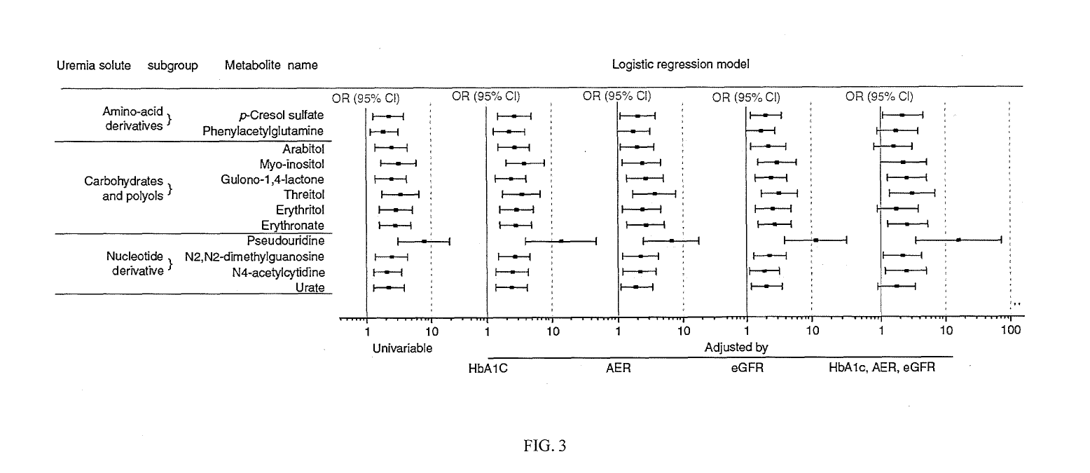

The fold difference between progressors and non-progressors and its q value for each of these metabolites are plotted in FIG. 2A according to whether the metabolite has been identified as a uremic solute. Among 119 metabolites that were common and stable over time within individual, 18 are known as uremic solutes and, of these, the fold difference was significantly increased above 1.0 for 12 (67%). To obtain a descriptive measure of their effects on the risk of progression to ESRD, logistic regression analysis was used to express the associations in terms of odds ratios for the outcome, ESRD. The results are grouped by biochemical class in FIG. 3, which also shows the odds ratios after adjustment for the clinical covariates, AER, eGFR and HbA1c. The fact that the associations remained after adjustment for AER, eGFR and HbA1c suggests that the effects of these uremic solutes are potentially independent from the clinical characteristics.

Among the amino acid-derived uremic solutes associated with progression to ESRD were two, p-cresol sulfate and phenylacetylglutamine, produced by the gut microbiome. Their effects on the risk of progression to ESRD were strong. For example, the odds ratio for progression to ESRD for a one standard deviation increase in plasma p-cresol sulfate concentration was 2.3 (95% CI; 1.3, 3.9) in univariable analysis. The effect of phenylacetylglutamine was similar, but slightly less than that of p-cresol sulfate. Of the six polyol derived uremic solutes significantly associated with the risk of progression to ESRD, myo-inositol was the one most strongly associated, odds ratio: 3.2 (95% CI; 1.7, 5.9). Of the four nucleotide-derived uremic solutes significantly associated with the risk of progression to ESRD, three are derived from degradation of RNA and the fourth (urate or uric acid) is derived from degradation of DNA. The strongest association with progression to ESRD was for pseudouridine, odds ratio 7.8 (95% CI; 3.1, 19).

Progression to ESRD According to Plasma Concentration of Amino Acids and their Derivatives:

Thirty-nine metabolites representing amino acids or their derivatives were common and stable over time and 14 of them (29%) were associated with risk of progression to ESRD. The fold difference between progressors and non-progressors and its q-value for each of these metabolites are plotted in FIG. 2B along with other common and stable metabolites after removal of the 16 uremic solutes (including 2 amino acid derivatives).

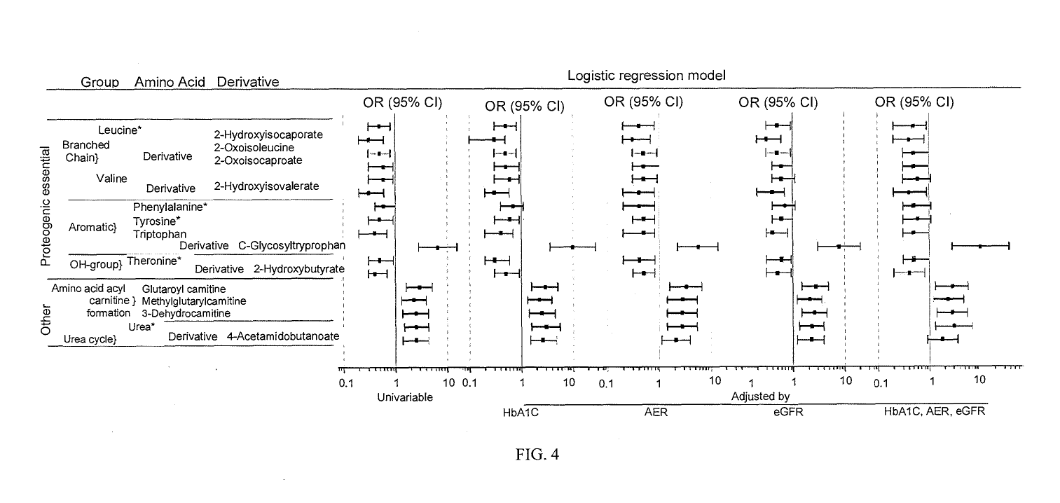

The effects of the remaining 12 amino acids on risk of ESRD were estimated with logistic regression FIG. 4. In addition, five essential amino acids are present, although they were not stable over time. As in FIG. 3, the odds ratios after adjustment for the group differences in AER, eGFR and HbA1c are also shown. It is important to note that none of the associations in FIG. 4 were diminished by the adjustments, therefore they seem to be independent of these clinical covariates. In contrast to the associations with uremic solutes, concentrations of many of these metabolites were higher in the non-progressors than the progressors. For example, low concentrations of six essential proteogenic amino acids were associated with progression to ESRD. The odds ratio for progression to ESRD for a one standard deviation increase in the plasma concentration of leucine was 0.5 (95% CI; 0.3, 0.8) and odds ratios for the remaining 5 amino acids were similar FIG. 4. In addition, five amino acid derivatives were negatively associated with risk of progression to ESRD. The odds ratio for a one standard deviation increase in plasma concentration of 2-hydroxyisocaproate (leucine derivative) was 0.3 (95% CI; 0.2, 0.6), and the odds ratios for the remaining five derivatives were similar.

A few amino acid derivatives were positively associated with progression to ESRD. C-glycosyltryptophan was elevated and the most significantly different between progressors and non-progressors among the metabolites shown in FIG. 2B. The odds ratio for a one standard deviation increase in its plasma concentration was 6.6 (95% CI; 2.8, 15). The five remaining metabolites, which are derivatives related to acylcarnitines and the urea cycle, were positively associated with progression to ESRD with smaller odds ratios.

Among other metabolites associated with ESRD risk, there were two lipids, dihomo-linolenate (20:3n3) and docosapentaenoate (n3 DPA; 22:5n3). Both are ω6 fatty acids and were negatively associated with risk of progression to ESRD. Gluconate and two metabolites involved in vitamin B metabolism: pantothenate and N1-methyl-2-pyridone-5-carboxamide were positively associated with progression to ESRD. Data for these five metabolites were not shown.

Reduction of Redundant Data

Among metabolites significantly associated with progression to ESRD, some may reflect on shared underlying biology. To evaluate the potential redundancy among these metabolites, a Spearman rank correlation matrix was created. Approximately two-thirds of the metabolites were strongly correlated (data not shown).

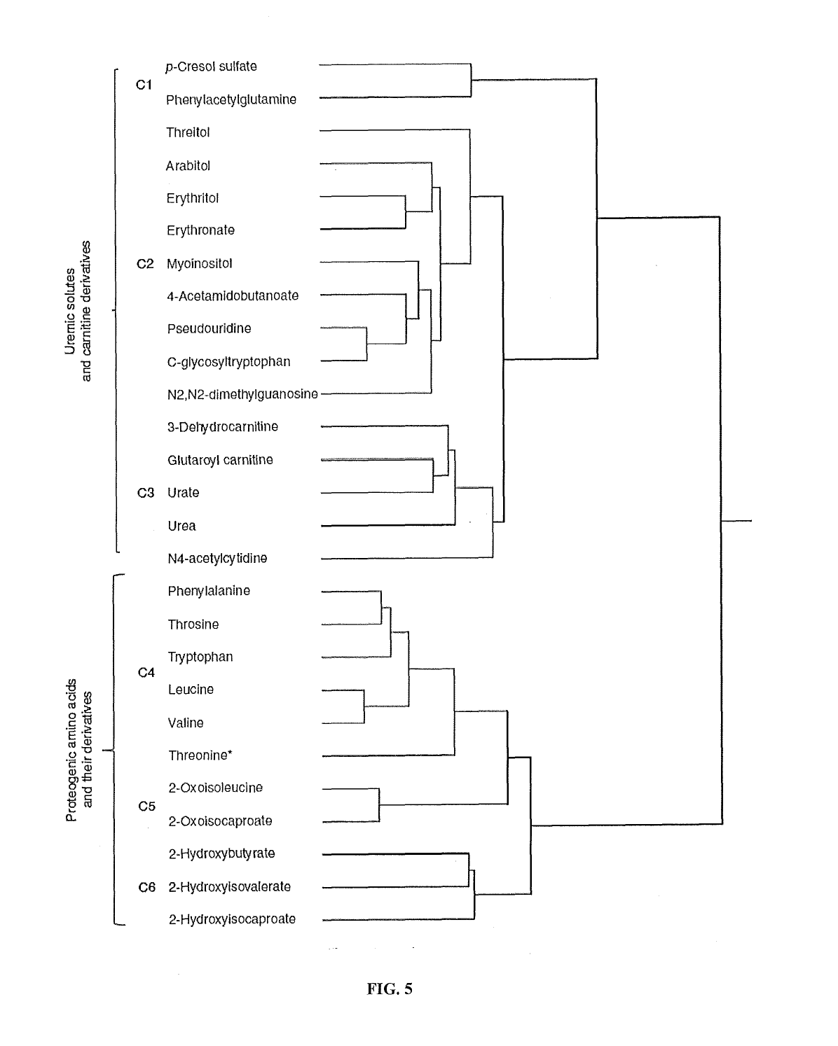

A cluster analysis revealed clusters (see FIG. 5) mirroring the patterns of our grouping based on the biological relevance presented in FIGS. 3 and 4, respectively.(28, 29) Clusters 1 and 2 comprised uremic solutes and C-glycosyltryptophan. Cluster 3 included carnitine derivatives, urate and urea. Cluster 4 comprised essential amino acids, cluster 5 their keto- and cluster 6 their hydroxylderivatives, respectively. In a logistic regression model including the leading metabolites from each cluster, erythritol, glutaryl carnitine and alphahydroxyisovalerate (from clusters 2, 3 and 6) remained significant. Odds ratios for an effect of one standard deviation difference in those metabolites considered together were: for erythritol 2.1 (95% CI 1.0, 4.5), for glutaroyl carnitine, 2.6 (95% CI 1.3, 5.4) and for 2-hydroxyisovalerate, 0.4 (95% CI 0.2, 0.9), respectively. Discrimination ability was c=0.89 for this model, while it was 0.74 to 0.75 for models including the significant metabolites separately. The p value for the difference tested by integrated discrimination improvement (IDI) test was p<0.001 for each single model in comparison with the model including all four metabolites. When either pseudouridine or C-glycosyltryptophan (the most significantly different metabolites between the progressors and non-progressors present in Cluster 2) were added to the logistic model with the representative of the other clusters, the effect of the remaining metabolites became borderline or non-significant.

Targeted Quantitative Metabolites Measurements

To validate these findings, targeted quantitative measurements of nine common and stable metabolites over time identified on the Metabolon global biochemical profiling platform was performed. Five out of nine metabolites were the ones strongly associated with progression to ESRD. Among the remaining four, two metabolites, indoxyl sulfate and hippurate were uremic solutes reported in the literature, but not significantly associated with progression in the global profiling study. The remaining two measured were allantoin and uracil, linked in the metabolic pathways related to urate and pseudouridine, respectively. Allantoin was not sufficiently detectable by the quantitative methods employed in this study (data not shown). For each of the other metabolites, association with progression to ESRD was consistent with the result obtained via global profiling. Also the association remained after adjustment for AER, eGFR and HbA1c (Table 3). Odds ratio for ESRD progression was 1.7 (95% CI; 1.0, 2.8) per one standard deviation of the logarithmically transformed metabolite, p-cresol sulfate and phenylacetylglutamine and higher than 2.5 for every other significant metabolite (pseudouridine, p-cresol sulfate, myoinositol and urate). It was confirmed that hippurate and indoxyl sulfate were not significantly associated with ESRD in this study. Logistic regression of the pseudouridine/uracil ratio did not improve the discrimination ability of the model (data not shown).

TABLE 3
Logistic regression analysis of the effect of plasma concentration of
uremic solutes measured by targeted quantitative metabolomics on
the risk of progression to ESRD in subjects with T2D.

| | | Logistic regression | |
|---|---|---|---|
| | | model univariable | |
| Uremic solute name | Mean ± s.d. (μmol/l) | OR (95% CI) | P-value |

Significant in the global profiling analysis

| | | | |
|---|---|---|---|
| p-Cresol sulfate | 33 ± 30 | 1.7 (1.0, 2.8) | 0.050 |
| Phenylacetylglutamine | 4 ± 5 | 1.7 (1.0, 2.9) | 0.041 |
| Myo-inositol | 52 ± 35 | 2.8 (1.5, 5.5) | 0.002 |
| Pseudouridine | 1.5 ± 0.8 | 2.4 (1.3, 4.3) | 0.004 |
| Urate | 275 ± 75^{a} | 2.5 (1.3, 4.8) | 0.007 |

Nonsignificant in the global profiling analysis

| | | | |
|---|---|---|---|
| Hippurate | 8.0 ± 8.5 | 0.9 (0.6, 1.5) | 0.798 |
| Indoxyl sulfate | 8.0 ± 9.3 | 1.3 (0.8, 2.1) | 0.280 |
| Uracil | 1.9 ± 6.8 | 1.3 (0.8, 2.0) | 0.355 |

Abbreviations:
CI, confidence interval;
ESRD, end-stage renal disease;
OR, odds ratios;
s.d., standard deviation;
T2D, type 2 diabetes.
Data are OR (95% CI) estimated as the effect per 1 s.d. of the logarithmically transformed metabolite.
^{a}To convert urate (uric acid) mmol/l concentrations to mg/dl, divide the values by 59.5. Concentration of the uric acid 275 mmol/l is equal to 4.6 mg/dl.

This study was performed in subjects with T2D, the majority of whom had normal renal function at baseline. Half progressed to ESRD and half did not during a decade of follow-up. In baseline plasma, progressors could be distinguished from non-progressors by high concentrations of metabolites referred to as uremic solutes and low concentrations of certain amino acids and their derivatives. This is the first demonstration that abnormal plasma concentrations of certain metabolites are associated with risk of progression to ESRD at a very early stage of diabetic nephropathy.

Uremic solutes, as catalogued by EUTox group,(7, 9) comprise compounds of different biochemical classes: amino acid derivatives, certain alcohol/polyols and modified nucleosides among them. Of the 18 known uremic solutes that were detected as common and stable metabolites with the Metabolon platform, 12 were elevated in subjects who progressed to ESRD. On the basis of results of multiple cross-sectional studies (7-10) the immediate interpretation of these findings might be that the increased concentration of uremic solutes was due to significant impairment of renal function in subjects who progressed to ESRD during 8-12 years of follow-up. However, the majority of progressors at baseline were in CKD stages 1 and 2 and had only slightly lower baseline eGFR than non-progressors (75±19 ml/min vs. 87±23 ml/min). In multivariable analyses, the odds ratios of progression to ESRD for specific uremic solutes were only minimally affected by adjustment for eGFR, AER and HbA1c.

Phenyl compounds, such as p-cresol sulfate and phenylacetylglutamine are the most extensively studied solutes known to increase in the uremic state.(8, 11, 13) These solutes can be toxic to endothelial cells and can contribute to increased risk of cardiovascular complications in patients with renal impairment.(30, 31) In humans these metabolites are exogenous and are produced by intestinal bacterial flora before they are absorbed into plasma and excreted through the kidney.(8, 11, 13, 32) Evidence confirming the microbiome as a source for these solutes was recently provided in a study of ESRD subjects with and without a colon.(11) In this study, high plasma concentrations of these solutes were associated with progression to ESRD.

Three additional solute derivatives of amino acids that are synthesized in the gut, phenol sulfate, indoleacetate and 3-indoxyl sulfate, were elevated in plasma of progressors compared to non-progressors, however, the differences did not reach statistical significance (see supplemental Table 2). All these findings support the notion that the gut microbiome (11, 13, 32) might control plasma levels of amino acid-derived uremic solutes, and their high levels increase the risk of progression to ESRD in subjects with T2D. The increase in circulating levels of these solutes also may be attributed to dietary factors.(34)

Elevated plasma concentrations of myo-inositol and other polyols were strongly associated with progression to ESRD in this study. These metabolites accumulate in plasma in the uremia state and during acute kidney injury.(9, 35, 36) The kidney is the most important organ for whole body metabolism of myo-inositol, as both the synthetic and degradative enzymes, L-myo-inositol-1-phosphate synthetase and myo-inositol oxygenase (MIOX), are highly expressed in the renal parenchyma.(37, 38) Myo-inositol can be obtained also from dietary sources. Regulation of its plasma levels is complex, involving glomerular filtration, reabsorption in proximal tubules in competition with glucose transport, apparent conversion to chiro-inositol in tissues and, finally, catabolic degradation of myo-inositol by MIOX, a protein that is upregulated by hyperglycemia.(36, 38-44) The high concentration gradients of myo-inositol in certain tissues such as kidney and the vascular endothelium are maintained by the osmoregulatory sodium/myo-inositol cotransporter 1 (SMIT1)(39, 40) Glucose/myo-inositol imbalance was demonstrated to induce proliferative and pro-fibrotic response in the proximal tubules in vitro and to alter the immune responses in leukocytes.(45, 46) Increased urinary excretion of myo-inositol and inositol imbalance in muscle tissue (high myo/chiro-inositol ratios) were reported in subjects with T2D.(42-44) In this study chiroinositol levels were hardly detectable (data not shown).

Plasma concentrations of several nucleotide derivatives that are considered to be uremic solutes were also strongly associated with progression to ESRD in this study. Among these derivatives, elevated concentration of pseudouridine in plasma was the strongest and most statistically significant predictor of progression. Pseudouridine belongs to the group of modified nucleosides that are regarded as indicators of whole-body RNA turnover.(47) These metabolites are increased in patients with malignancies,(48) and with uremia.(8, 49-51) Pseudouridine is synthesized from uracil(52, 53) and constitutes an end product, as it is not catabolized in humans.(54) Trace study in humans with radiolabeled pseudouridine showed that the kidney handling includes glomerular filtration and also tubular reabsorption.(55) High plasma levels of two other pyrimidine derivatives that are closely correlated with plasma levels of pseudouridine also increased the risk of ESRD progression (N2, N2-dimethylguanosine, and N4-acetylcytidine). These three nucleotides increase in plasma as urinary excretion decreases and accumulate significantly in the uremic state.(51)

Urate (uric acid) is a metabolite of purine metabolism. In this study, increased plasma concentrations were associated with progression to ESRD. Urate is another compound known to accumulate in the uremic state.(8, 9) Its increase, however, is disproportionally small due to compensatory mechanisms including increased enteric excretion, decreased production(56) and possibly altered tubular handling.(57) In a recent study, elevated plasma level of urate was a strong predictor of early renal function decline during follow-up of a large cohort of subjects with T1D.(58)

Kidney protein turnover, as compared with muscle and splanchnic turnover, is characterized by the highest rates of protein synthesis and amino acid oxidation, mainly in the tubulointerstitium.(17, 66) Depletion of the circulating pools of branched chain amino acids and tryptophan are known phenomena accompanying advanced chronic kidney disease.(10, 16, 17) On the other hand, increase in those amino acids was shown to predict development of T2D. (19) Interestingly, this study revealed that not only branched and aromatic, but also all other essential amino acids and their derivatives were lower in subjects who progressed to ESRD than in those who were non-progressors. In an experimental model of acute kidney injury, one of the strongest metabolic responses to nephrotoxins was massive excretion of all essential amino acids.(35, 67) It needs to be determined whether impaired tubular reabsorption contributes to the decreased levels of amino acids in early diabetic nephropathy. Amino acids that were decreased in subjects at risk of ESRD in this study all have a neutral charge. They are handled by the B0AT1 cotransporter responsible for the luminal influx, the heterodimeric exchanger and the facilitated diffusion transporter TAT1 in charge of the basolateral efflux and possibly by other transporters.(68, 69)

Among amino-acid derivatives, C-glycosyltryptophan showed a different pattern of association than the others. Its plasma concentration was the highest in progressors when compared with non-progressors. After pseudouridine it had the second highest fold difference between the study groups. Plasma concentrations of both were very highly correlated. Interestingly, they both carry a C-glycosylation linkage, a rare type of posttranslational protein modification.(70) Increased expression of the proteins containing certain forms of C-glycosylated tryptophan in the aortic vessels have been reported in the diabetic rats.(71) C-glycosylated tryptophan also correlates with eGFR.(72)

Analysis of acylcarnitines revealed that the increased concentrations were independently associated with risk of progression to ESRD. Acylcarnitines are filtered through the kidney and about 75% are excreted into urine.(73) Serum acylcarnitines deriving from lipid and amino acids are inversely correlated with GFR in individuals with normal as well as with impaired renal function.(18, 74, 75) Acylcarnitines transport is regulated by organic carnitine transporters in the kidney.(61) In this study, amino acid-deriving (but not lipid-deriving) acylcarnitines were increased in the subjects at risk. They are generated via beta-oxidation of the branched chain amino acids. Those amino acids and their intermediate keto acid derivatives were also depleted in this study (2-oxoisoleucine, 2-oxoisocaproate).

Supplemental Tables 1A-1D.

Analytical and intraindividual performance of the 445 metabolites detected by global metabolomic profiling in the study subjects stratified by the biochemical classes (1A—amino acids, 1B—carbohydrates, 1C—lipids, 1D—metabolites that belong to other than the major three classes. Metabolites are stratified by their detectability and subsequently presented in the alphabetic order. Drug related metabolites are not displayed.

SUPPLEMENTAL TABLE 1A

| | | intraindividual |
|---|---|---|
| | detection | rank correlation |
| | frequency | coefficient |
| Biochemical class/metabolite name | [%] | r |
| Amino acids | | |
| 2-aminobutytate | 100 | 0.54 |
| 2-hydroxybutyrate | 100 | 0.83 |
| 2-methylbutyroylcarnitine | 99 | 0.41 |
| 3-(4-hydroxyphenyl)lactate | 100 | 0.53 |
| 3-dehydrocarnitine | 100 | 0.61 |
| 3-hydroxyisobutyrate | 100 | 0.71 |
| 3-indoxyl sulfate | 100 | 0.73 |
| 3-methoxytyrosine | 96 | 0.00 |
| 3-methyl-2-oxobutyrate | 89 | 0.00 |
| 3-methyl-2-oxovalerate | 100 | 0.47 |
| 3-methylhistidine | 100 | 0.24 |
| 3-phenylpropionate (hydrocinnamate) | 79 | 0.55 |
| 4-acetamidobutanoate | 100 | 0.76 |
| 4-methyl-2-oxopentanoate | 100 | 0.75 |
| 5-oxoproline | 100 | 0.59 |
| alanine | 100 | 0.28 |
| alpha-hydroxyisocaproate | 88 | 0.44 |
| alpha-hydroxyisovalerate | 100 | 1.00 |
| arginine | 95 | 0.52 |
| asparagine | 99 | 0.00 |
| aspartate | 100 | 0.33 |
| beta-hydroxypyruvate | 75 | 0.50 |
| betaine | 100 | 0.82 |
| C-glycosyltryptophan | 100 | 0.64 |
| citrulline | 100 | 0.42 |
| creatine | 100 | 0.33 |
| creatinine | 100 | 0.35 |
| dimethylglycine | 98 | 0.00 |
| glutamate | 100 | 0.52 |
| glutamine | 100 | 0.00 |
| glutaroyl camitine | 100 | 0.54 |
| glycine | 100 | 0.77 |
| histidine | 100 | 0.26 |
| indoleacetate | 99 | 0.89 |
| indolelactate | 100 | 0.55 |
| indolepropionate | 100 | 0.54 |
| isobutyrylcamitine | 100 | 0.45 |
| isoleucine | 100 | 0.19 |
| isovalerylcarnitine | 100 | 0.68 |
| kynurenate | 83 | 0.00 |
| kynurenine | 100 | 0.35 |
| leucine | 100 | 0.16 |
| levulinate (4-oxovalerate) | 100 | 0.65 |
| lysine | 100 | 0.09 |
| methionine | 100 | 0.12 |
| methylglutaroylcarnitine | 100 | 0.78 |
| N-acetylalanine | 100 | 0.26 |
| N-acetyl-beta-alanine | 100 | 0.02 |
| N-acetylglycine | 86 | 0.26 |
| N-acetylmethionine | 100 | 0.35 |
| N-formylmethionine | 100 | 0.33 |
| omithine | 98 | 0.09 |
| p-cresol sulfate | 100 | 0.78 |
| phenol sulfate | 100 | 0.78 |
| phenylacetate | 76 | 0.00 |
| phenylacetylglutamine | 100 | 0.81 |
| phenylalanine | 100 | 0.31 |
| phenyllactate | 91 | 0.31 |
| pipecolate | 100 | 0.41 |
| proline | 100 | 0.00 |
| pyroglutamine | 98 | 0.38 |
| serine | 100 | 0.64 |
| serotonin (5HT) | 74 | 0.89 |
| threonine | 100 | 0.35 |
| tiglyl camitine | 100 | 0.27 |
| trans-4-hydroxyproline | 98 | 0.00 |
| tryptophan | 100 | 0.54 |
| tryptophan betaine | 100 | 0.70 |
| tyrosine | 100 | 0.04 |
| urea | 100 | 0.28 |
| valine | 100 | 0.50 |
| Amino acids detected in less than two third | | |
| of the study subjects | | |
| 2-hydroxy-3-methylvalerate | 21 | na |
| 3-(3-hydroxyphenyl)propionate | 15 | na |
| 3-hydroxy-2-ethylpropionate | 59 | na |
| 4-hydroxyphenylacetate | 26 | na |
| 4-hydroxyphenylpyruvate | 29 | na |
| 5-methylthiadenosine | 41 | na |
| alpha-ketobutyrate | 36 | na |
| beta-alanine | 61 | na |
| beta-hydroxyisovalerate | 63 | na |
| cysteine | 38 | na |
| gentisate | 11 | na |
| hydroxyisovaleroyl carnitine | 55 | na |
| isovalerylglycine | 14 | na |
| N6-acetyllysine | 60 | na |
| N-acetylomithine | 63 | na |
| N-acetylphenylalanine | 60 | na |
| N-acetylthreonine | 59 | na |
| N-acetylvaline | 24 | na |
| N-methyl proline | 53 | na |
| o-cresol sulfate | 43 | na |
| spermine | 0 | na |

SUPPLEMENTAL TABLE 1B

| | | intraindividual |
|---|---|---|
| | detection | rank correlation |
| | frequency | coefficient |
| Biochemical class/metabolite name | [%] | r |
| Carbohydrates and Polyols | | |
| 1,5-anhydroglucitol | 89 | 0.58 |
| 1,6-anhydroglucose | 95 | 0.63 |
| arabinose | 96 | 0.26 |
| arabitol | 100 | 0.43 |
| erythritol | 100 | 0.49 |
| erythronate | 100 | 0.47 |
| fructose | 100 | 0.45 |
| fucose | 93 | 0.70 |
| gluconate | 100 | 0.65 |
| glucose | 100 | 0.71 |
| glycerate | 100 | 0.42 |
| gulono-1,4-lactone | 81 | 0.39 |
| lactate | 100 | 0.00 |
| mannitol | 96 | 0.00 |
| mannose | 100 | 0.72 |
| myo-inositol | 100 | 0.49 |
| pyruvate | 100 | 0.41 |
| ribitol | 88 | 0.00 |
| threitol | 100 | 0.54 |
| xylonate | 96 | 0.07 |
| Carbohydrates and Polyols detected in less | | |
| than two third of the study subjects | | |
| 3-phosphoglycerate | 34 | na |
| maltose | 33 | na |
| oxalate (ethanedioate) | 16 | na |
| sorbitol | 49 | na |
| sucrose | 21 | na |
| trehalose | 46 | na |
| xylitol | 48 | na |

SUPPLEMENTAL TABLE 1C

| | | intraindividual |
|---|---|---|
| | detection | rank correlation |
| | frequency | coefficient |
| Biochemical class/metabolite name | [%] | r |
| Lipids | | |
| 1,2-propanediol | 100 | 0.00 |
| 10-heptadecenoate(17:1n7) | 100 | 0.59 |
| 10-nonadecenoate (19:1n9) | 100 | 0.04 |
| 13-HODE + 9-HODE | 100 | 0.14 |
| 13-methylmyristic acid | 100 | 0.00 |
| 15-HETE | 89 | 0.00 |
| 17-methylstearate | 99 | 0.66 |
| 1-arachidonoylglycerophosphocholine | 100 | 0.00 |
| 1-arachidonoylglycerophosphoethanolamine | 100 | 0.37 |
| 1-arachidonoylglycerophosphoinositol | 100 | 0.00 |
| 1-docosahexaenoylglycerophosphocholine | 100 | 0.28 |
| 1-docosapentaenoylglycerophosphocholine | 93 | 0.01 |
| 1-eicosadienoylglycerophosphocholine | 93 | 0.00 |
| 1-eicosatrienoylglycerophosphocholine | 100 | 0.00 |
| 1-heptadecanoylglycerophosphocholine | 100 | 0.00 |
| 1-linoleoylglycerophosphocholine | 100 | 0.18 |
| 1-linoleoylglycerophosphoethanolamine | 100 | 0.24 |
| 1-myristoylglycerophosphocholine | 100 | 0.08 |
| 1-oleoylglycerol (1-monoolein) | 98 | 0.24 |
| 1-oleoylglycerophosphocholine | 100 | 0.00 |
| 1-oleoylglycerophosphoethanolamine | 100 | 0.66 |
| 1-palmitoleoylglycerophosphocholine | 100 | 0.00 |
| 1-palmitoylglycerol (1-monopalmitin) | 100 | 0.14 |
| 1-palmitoylglycerophosphocholine | 100 | 0.00 |
| 1-palmitoylglycerophosphoethanolamine | 100 | 0.01 |
| 1-palmitoylglycerophosphoinositol | 83 | 0.15 |
| 1-pentadecanoylglycerophosphocholine | 100 | 0.00 |
| 1-stearoylglycerol (1-monostearin) | 100 | 0.00 |
| 1-stearoylglycerophosphocholine | 100 | 0.00 |
| 1-stearoylglycerophosphoethanolamine | 100 | 0.00 |
| 1-stearoylglycerophosphoinositol | 100 | 0.67 |
| 21-hydroxypregnenolone disulfate | 89 | 0.78 |
| 2-arachidonoylglycerophosphocholine | 94 | 0.18 |
| 2-arachidonoylglycerophosphoethanolamine | 96 | 0.02 |
| 2-hydroxypalmitate | 100 | 0.10 |
| 2-hydroxystearate | 100 | 0.00 |
| 2-linoleoylglycerophosphocholine | 90 | 0.21 |
| 2-linoleoylglycerophosphoethanolamine | 95 | 0.15 |
| 2-oleoylglycerophosphocholine | 100 | 0.00 |
| 2-oleoylglycerophosphoethanolamine | 83 | 0.51 |
| 2-palmitoylglycerophosphocholine | 100 | 0.00 |
| 2-palmitoylglycerophosphoethanolamine | 90 | 0.00 |
| 2-stearoylglycerophosphocholine | 100 | 0.00 |
| 3-carboxy-4-methyl-5-propyl-2-furanpropanoate | 100 | 0.84 |
| 3-hydroxybutyrate | 100 | 0.64 |
| 3-hydroxydecanoate | 96 | 0.68 |
| 4-androsten-3beta,17beta-diol disulfate 1 | 100 | 0.84 |
| 4-androsten-3beta,17beta-diol disulfate 2 | 100 | 0.76 |
| 5alpha-androstan-3beta,17beta-diol disulfate | 91 | 0.87 |
| 5alpha-pregnan-3beta,20alpha-diol disulfate | 85 | 0.63 |
| 7-alpha-hydroxy-3-oxo-4-cholestenoate | 100 | 0.47 |
| acetylcarnitine | 100 | 0.42 |
| adrenate (22:4n6) | 100 | 0.24 |
| andro steroid monosulfate 2 | 100 | 0.82 |
| androsterone sulfate | 94 | 0.92 |
| arachidonate (20:4n6) | 100 | 0.00 |
| azelate (nonanedioate) | 100 | 0.10 |
| butyrylcarnitine | 96 | 0.37 |
| caprate (10:0) | 100 | 0.81 |
| caproate (6:0) | 100 | 0.30 |
| caprylate (8:0) | 100 | 0.37 |
| carnitine | 100 | 0.78 |
| cholate | 79 | 0.38 |
| cholesterol | 100 | 0.35 |
| choline | 100 | 0.28 |
| cis-4-decenoyl carnitine | 100 | 0.24 |
| cis-vaccenate (18:1n7) | 99 | 0.31 |
| cortisol | 100 | 0.00 |
| cortisone | 100 | 0.12 |
| decanoylcarnitine | 99 | 0.05 |
| dehydroisoandrosterone sulfate (DHEA-S) | 100 | 0.83 |
| deoxycarnitine | 100 | 0.00 |
| dihomo-linoleate (20:2n6) | 100 | 0.24 |
| dihomo-linolenate (20:3n3 or n6) | 100 | 0.59 |
| docosadienoate (22:2n6) | 98 | 0.52 |
| docosahexaenoate (DHA; 22:6n3) | 100 | 0.66 |
| docosapentaenoate (n3 DPA; 22:5n3) | 100 | 0.47 |
| docosapentaenoate (n6 DPA; 22:5n6) | 100 | 0.00 |
| eicosanodioate | 100 | 0.71 |
| eicosapentaenoate (EPA; 20:5n3) | 100 | 0.36 |
| eicosenoate (20:1n9 or 11) | 100 | 0.35 |
| epiandrosterone sulfate | 96 | 0.87 |
| glycerol | 99 | 0.54 |
| glycerol 3-phoshate (G3P) | 100 | 0.12 |
| glycerophosphorylcholine (GPC) | 96 | 0.31 |
| glycochenodeoxycholate | 98 | 0.62 |
| glycocholate | 100 | 0.52 |
| glycocholenate sulfate | 94 | 0.67 |
| glycodeoxycholate | 95 | 0.10 |
| glycolithocholate sulfate | 99 | 0.12 |
| glycoursodeoxycholate | 96 | 0.50 |
| heptanoate (7:0) | 100 | 0.77 |
| hexadecanedioate | 99 | 0.89 |
| hexanoylcarnitine | 100 | 0.72 |
| lathosterol | 73 | 0.00 |
| laurate (12:0) | 100 | 0.58 |
| leukotriene B4 | 80 | 0.00 |
| linoleate (18:2n6) | 100 | 0.68 |
| linolenate [alpha or gamma; (18:3n3 or 6)] | 100 | 0.77 |
| margarate (17:0) | 100 | 0.28 |
| mead acid (20:3n9) | 96 | 0.00 |
| myristate (14:0) | 100 | 0.70 |
| myristoleate (14:1n5) | 100 | 0.62 |
| nonadecanoate (19:0) | 100 | 0.33 |
| octadecanedioate | 98 | 0.72 |
| octanoylcarnitine | 100 | 0.19 |
| oleate (18:1n9) | 100 | 0.68 |
| oleoylcarnitine | 100 | 0.16 |
| palmtitate (16:0) | 100 | 0.66 |
| palmitoleate (16:1n7) | 100 | 0.38 |
| palmitoyl sphingomyelin | 100 | 0.37 |
| palmitoylcarnitine | 99 | 0.04 |
| pelargonate (9:0) | 100 | 0.50 |
| pentadecanoate (15:0) | 98 | 0.04 |
| pregn steroid monosulfate | 100 | 0.84 |
| pregnen-diol disulfate | 100 | 0.92 |
| pregnenolone sulfate | 95 | 0.73 |
| propionylcarnitine | 100 | 0.03 |
| sebacate (decanedioate) | 100 | 0.00 |
| sphingosine | 90 | 0.00 |
| stearate (18:0) | 100 | 0.37 |
| stearidonate (18:4n3) | 95 | 0.81 |
| stearoyl sphingomyelin | 100 | 0.28 |
| stearoylcarnitine | 96 | 0.55 |
| taurocholate | 84 | 0.73 |
| taurocholenate sulfate | 91 | 0.56 |
| taurolithocholate 3-sulfate | 89 | 0.36 |
| tetradecanedioate | 99 | 0.93 |
| undecanoate (11:0) | 100 | 0.21 |
| Lipids detected in less than two third of the study subjects | | |
| 10-undecenoate (11:1n1) | 63 | na |
| 12,13-epoxy-9-keto-10(trans)-octadecenoate | 31 | na |
| 15-methylpalmitate (isobar with 2-methylpalmitate) | 38 | na |
| 15-oxo-5Z,8Z,11Z,13E-eicosatetraenoate | 33 | na |
| 16-hydroxypalmitate | 63 | na |
| 1-arachidoylglycerophosphocholine | 54 | na |
| 1-linoleoylglycerol (1-monolinolein) | 63 | na |
| 1-palmitoylplasmenylethanolamine | 63 | na |
| 2-docosahexaenoylglycerophosphocholine | 38 | na |
| 2-docosahexaenoylglycerophosphoethanolamine | 34 | na |
| 2-hydroxyglutarate | 60 | na |
| 2-hydroxyoctanoate | 60 | na |
| 2-linoleoylglycerol (2-monolinolein) | 35 | na |
| 2-myristoylglycerophosphocholine | 66 | na |
| 2-octenoyl carnitine | 61 | na |
| 2-palmitoleoylglycerophosphocholine | 36 | na |
| 3-hydroxyoctanoate | 33 | na |
| 5alpha-androstan-3alpha,17beta-diol disulfate | 41 | na |
| 5alpha-androstan-3beta,17alpha-diol disulfate | 28 | na |
| 5-dodecenoate (12:1n7) | 38 | na |
| 5-HEPE | 1 | na |
| 5-HETE | 65 | na |
| 5-oxoETE | 14 | na |
| 1-alpha-hydroxycholesterol | 33 | na |
| 7-beta-hydroxycholesterol | 46 | na |
| chiro-inositol | 48 | na |
| deoxycholate | 63 | na |
| dihydrocholesterol | 35 | na |
| docosadioate | 34 | na |
| isovalerate | 63 | na |
| laurylcarnitine | 60 | na |
| methyl palmitate (15 or 2) | 63 | na |
| prostaglandin A2 | 39 | na |
| prostaglandin B2 | 20 | na |
| prostaglandin E2 | 38 | na |
| taurochenodeoxycholate | 34 | na |
| taurodeoxycholate | 35 | na |
| testosterone | 1 | na |
| undecanedioate | 63 | na |
| valerate | 61 | na |
| valerylcarnitine | 43 | na |

SUPPLEMENTAL TABLE 1D

| | | intraindividual |
|---|---|---|
| | detection | rank correlation |
| | frequency | coefficient |
| Biochemical class/metabolite name | [%] | r |
| Nucleotides | | |
| 5-methyluridine (ribothymidine) | 80 | 0.43 |
| adenosine 5′-monophosphate | 98 | 0.59 |
| allantoin | 98 | 0.02 |
| hypoxanthine | 96 | 0.00 |
| inosine | 88 | 0.50 |
| N1-methyladenosine | 100 | 0.32 |
| N2,N2-dimethylguanosine | 98 | 0.82 |
| N4-acetylcytidine | 86 | 0.86 |
| N6-carbamoylthreonyladenosine | 96 | 0.16 |
| pseudouridine | 100 | 0.68 |
| urate | 100 | 0.68 |
| uridine | 100 | 0.15 |
| xanthine | 99 | 0.58 |
| Nucleotides detected in less than two third | | |
| of the study subjects | | |
| 5,6-dihydrouracil | 35 | na |
| adenosine 2′-monophosphate | 28 | na |
| N1-methylguanosine | 54 | na |
| N6-methyladenosine | 60 | na |
| uracil | 63 | na |
| Varia | | |
| 1,7-dimethylurate | 89 | 0.98 |
| 2-hydroxyhippurate (salicylurate) | 93 | −0.01 |
| 3-hydroxyhippurate | 90 | 0.62 |
| 3-methylxanthine | 69 | 0.81 |
| 4-hydroxyhippurate | 93 | 0.25 |
| acetylphosphate | 100 | 0.24 |
| aplha-ketoglutarate | 90 | 0.04 |
| aplha-tocopherol | 98 | 0.00 |
| benzoate | 100 | 0.04 |
| beta-tocopherol | 88 | 0.00 |
| bilirubin (E,E) | 99 | 0.16 |
| biliverdin | 99 | 0.19 |
| bradykinin | 99 | 0.37 |
| bradykinin, des-arg(9) | 95 | 0.35 |
| caffeine | 99 | 0.84 |
| catechol sulfate | 100 | 0.81 |
| cinnamoylglycine | 69 | 0.63 |
| citrate | 100 | 0.39 |
| complement 3 fragment: HWESASXX | 98 | 0.00 |
| complement 3 fragment: XHWESASXXR | 98 | 0.04 |
| ergothioneine | 80 | 0.26 |
| fibrinopeptide A fragment: | 75 | 0.00 |
| ADpSGEGDFXAEGGGVR | | |
| fibrinopeptide A fragment: | 100 | 0.32 |
| ADSGEGDFXAEGGGVR | | |
| fumarate | 100 | 0.05 |
| gamma-glutamylisoleucine | 100 | 0.12 |
| gamma-glutamylleucine | 100 | 0.10 |
| gamma-glutamylmethionine | 94 | 0.09 |
| gamma-glutamylphenylalanine | 100 | 0.18 |
| gamma-glutamyltyrosine | 99 | 0.21 |
| gamma-glutamylvaline | 100 | 0.00 |
| heme | 98 | 0.00 |
| hippurate | 100 | 0.62 |
| homostachydrine | 98 | 0.28 |
| malate | 100 | 0.14 |
| N-(2-furoyl)glycine | 75 | 0.79 |
| nicotinamide | 100 | 0.00 |
| paraxanthine | 98 | 0.90 |
| phosphate | 100 | 0.31 |
| piperine | 94 | 0.18 |
| pyridoxate | 100 | 0.25 |
| stachydrine | 100 | 0.52 |
| succinylcarnitine | 100 | 0.25 |
| tartarate | 76 | −0.81 |
| theobromine | 98 | 0.70 |
| theophylline | 91 | 0.91 |
| thymol sulfate | 68 | 0.87 |
| xylose | 84 | 0.67 |
| Various metabolites in less than two third | | |
| of the study subjects | | |
| 1,3,7-trimethylurate | 58 | na |
| 1,3-dimethylurate | 9 | na |
| 1-methylurate | 51 | na |
| 1-methylxanthine | 46 | na |
| 2,3-dihydroxyisovalerate | 15 | na |
| 3-ethylphenylsulfate | 25 | na |
| 4-ethylphenylsulfate | 34 | na |
| 4-vinylphenol sulfate | 63 | na |
| 5-acetylamino-6-amino-3-methyluracil | 61 | na |
| 7-methylxanthine | 46 | na |
| alpha-CEHC glucuronide | 6 | na |
| alpha-glutamyltryptophan | 1 | na |
| arabonate | 36 | na |
| bilirubin (E,Z or Z,E) | 51 | na |
| cis-aconitate | 38 | na |
| dihydroferulic acid | 19 | na |
| fibrinogen alpha chain: ADDTWEPFASGK | 6 | na |
| galacturonate | 31 | na |
| HXGXA | 46 | na |
| isoleucylvaline | 58 | na |
| leucylalanine | 24 | na |
| leucylhistidine | 3 | na |
| leucylphenylalanine | 50 | na |
| methionylalanine | 16 | na |
| phenylalanylphenylalanine | 16 | na |
| pro-hydroxy-proline | 63 | na |
| pyrophosphate (PPi) | 65 | na |
| quinate | 35 | na |
| saccharin | 60 | na |
| Thymosin β4 fragment: Ac-Ser-Asp-Lys- | 14 | na |
| Pro-OH | | |

SUPPLEMENTAL TABLE 2
Analytical and intraindividual performance of the common uremic solutes in the global metabolomic
profiling in the study subjects. Poorly detectable uremic solutes are not listed.

| | | | | intraindividual | | | |
|---|---|---|---|---|---|---|---|
| | colon- | circulating | detection | rank correlation | fold | | postive false |
| Uremic solute | derived | fraction | frequency | coefficient | change | signficance | discovery rate |
| metabolite name | yes | type | [%] | r | case/ctrl | p | q |
| Amino acid derivatives | | | | | | | |
| Phenylalanine derivatives | | | | | | | |
| p-cresol sulfate | yes | bound | 100 | 0.8 | 1.3 | 0.001 | 0.005 |
| phenol sulfate | yes | bound | 100 | 0.8 | 1.2 | 0.131 | 0.146 |
| phenylacetate | | soluble | 76 | −0.1 | 1.0 | 0.613 | 0.331 |
| phenylacetylglutamine | yes | soluble | 100 | 0.8 | 1.2 | 0.006 | 0.019 |
| Tryptophan derivatives | | | | | | | |
| kynurenate | | bound | 83 | −0.1 | 1.2 | 0.044 | 0.078 |
| indoleacetate | yes | bound | 99 | 0.9 | 1.2 | 0.182 | 0.170 |
| 3-indoxyl sulfate | yes | bound | 100 | 0.7 | 1.2 | 0.182 | 0.170 |
| indolelactate | | bound | 100 | 0.6 | 1.0 | 0.813 | 0.388 |
| indolepropionate | yes | bound | 100 | 0.5 | 0.9 | 0.043 | 0.078 |
| Glycine/Serine derivatives | | | | | | | |
| cinnamoylglycine | yes | soluble | 69 | 0.6 | 1.0 | 0.862 | 0.398 |
| hippurate | yes | bound | 100 | 0.6 | 1.0 | 0.605 | 0.331 |
| dimethylglycine | yes | soluble | 98 | −0.2 | 1.3 | 0.001 | 0.006 |
| Nucleotide derivatives | | | | | | | |
| Purine derivatives | | | | | | | |
| hypoxanthine | | soluble | 96 | −0.1 | 0.9 | 0.085 | 0.115 |
| N6-carbamoylthreonyladenosine | | soluble | 96 | 0.2 | 1.5 | 2 × 10⁻⁵ | 1 × 10⁻⁴ |
| N2,N2-dimethylguanosine | | soluble | 98 | 0.8 | 1.3 | 0.000 | 0.003 |
| xanthine | | soluble | 99 | 0.6 | 0.9 | 0.868 | 0.399 |
| N1-methyladenosine | | soluble | 100 | 0.3 | 1.3 | 0.000 | 0.002 |
| urate | | soluble | 100 | 0.7 | 1.3 | 0.001 | 0.006 |
| Pyrimidine derivative | | | | | | | |
| N4-acetylcytidine | | soluble | 86 | 0.9 | 1.3 | 0.003 | 0.009 |
| pseudouridine | | soluble | 100 | 0.7 | 1.5 | 1 × 10⁻⁹ | 1 × 10⁻⁷ |
| uridine | | soluble | 100 | 0.2 | 0.9 | 0.266 | 0.215 |
| Alcohols and polyols | | | | | | | |
| myo-inositol | | soluble | 100 | 0.5 | 1.4 | 1 × 10⁻⁵ | 0.0004 |
| arabitol | | soluble | 100 | 0.4 | 1.3 | 3 × 10⁻⁴ | 0.003 |
| threitol | | soluble | 100 | 0.5 | 1.3 | 7 × 10⁻⁷ | 2 × 10⁻⁴ |
| erythritol | yes | soluble | 100 | 0.5 | 1.3 | 6 × 10⁻⁵ | 0.001 |
| Nicotinamide derivative | | | | | | | |
| N1-Methyl-2-pyridone-5-carboxamide | | soluble | 99 | 0.6 | 1.3 | 0.004 | 0.013 |
| nicotinamide | yes | soluble | 100 | −0.7 | 0.9 | 0.035 | 0.068 |

SUPPLEMENTAL TABLE 3
Comparison of platform performance between amino acids measurements performed by global
metabolomic profiling (Metabolon Inc) and quantitative measurements performed with
gas chromatography-mass spectroscopy (GC-MS) in the University of Michigan (UM).

| | Metabolon Inc | | | | |
|---|---|---|---|---|---|
| | semiquantitive | | U-M (GC/MS) | | comparison between |
| | | intraindividual | quantitive | | the two platforms |
| | detection | rank correlation | detection | absolute | interplatform rank |
| platform | frequency | coefficient | frequency | concentrations | correlation coefficient |
| measurements type | [%] | r | [%] | μM | r |
| alanine | 100 | 0.28 | 100 | 524 ± 131 | 0.57 |
| asparagine | 99 | 0.00 | 100 | 27 ± 12 | 0.70 |
| aspartic acid | 100 | 0.33 | 100 | 17 ± 11 | 0.91 |
| glutamic acid | 100 | 0.52 | 100 | 339 ± 216 | 0.93 |
| glutamine | 100 | 0.00 | 100 | 189 ± 478 | 0.73 |
| glycine | 100 | 0.77 | 100 | 211 ± 76 | 0.76 |
| histidine | 100 | 0.26 | 100 | 53 ± 31 | 0.58 |
| hydroxyproline | 98 | 0.00 | 65 | 2.3 ± 3.6 | 0.15 |
| isoleucine | 100 | 0.19 | 100 | 85 ± 40 | 0.84 |
| lucine | 100 | 0.16 | 100 | 160 ± 69 | 0.85 |
| lysine | 100 | 0.09 | 100 | 186 ± 68 | 0.13 |
| methionine | 100 | 0.12 | 100 | 14 ± 9 | 0.88 |
| ornithine | 98 | 0.09 | 100 | 57 ± 25 | 0.42 |
| phenylalanine | 100 | 0.31 | 100 | 57 ± 19 | 0.74 |
| proline | 100 | 0.00 | 100 | 234 ± 74 | 0.87 |
| serine | 100 | 0.64 | 100 | 69 ± 44 | 0.49 |
| threonine | 100 | 0.35 | 100 | 76 ± 36 | 0.71 |
| tryptophan | 100 | 0.54 | 100 | 21 ± 12 | 0.48 |
| tyrosine | 100 | 0.04 | 100 | 51 ± 22 | 0.86 |
| valine | 100 | 0.50 | 100 | 328 ± 107 | 0.72 |

### REFERENCE LIST

1. U.S. Renal Data System, USRDS 2010 Annual Data Report: Atlas of Chronic Kidney Disease and End-Stage Renal Disease in the United States, National Institutes of Health, National Institute of Diabetes and Digestive and Kidney Diseases, Bethesda, Md., 2010. 2010
2. Wang J H, Byun J, Pennathur S. Analytical approaches to metabolomics and applications to systems biology. Semin Nephrol 2010; 30:500-11.
3. Portilla D, Schnackenberg L, Beger R D. Metabolomics as an extension of proteomic analysis: study of acute kidney injury. Semin Nephrol 2007; 27:609-20.
4. Dehaven C D, Evans A M, Dai H, Lawton K A. Organization of GC/MS and LC/MS metabolomics data into chemical libraries. J Cheminform 2010; 2:9.
5. Evans A M, Dehaven C D, Barrett T, Mitchell M, Milgram E. Integrated, nontargeted ultrahigh performance liquid chromatography/electrospray ionization tandem mass spectrometry platform for the identification and relative quantification of the small-molecule complement of biological systems. Anal Chem 2009; 81:6656-67.
6. Ohta T, Masutomi N, Tsutsui N, et al. Untargeted metabolomic profiling as an evaluative tool of fenofibrate-induced toxicology in Fischer 344 male rats. Toxicol Pathol 2009; 37:521-35.
7. Duranton F, Cohen G, De Smet R, et al. Normal and pathologic concentrations of uremic toxins. J Am Soc Nephrol 2012; 23:1258-70.
8. Meyer T W, Hostetter T H. Uremia. N Engl J Med 2007; 357:1316-25.
9. Vanholder R, De Smet R, Glorieux G, et al. Review on uremic toxins: classification, concentration, and interindividual variability. Kidney Int 2003; 63:1934-43.
10. Rhee E P, Souza A, Farrell L, et al. Metabolite profiling identifies markers of uremia. J Am Soc Nephrol 2010; 21:1041-51.
11. Aronov P A, Luo F J, Plummer N S, et al. Colonic contribution to uremic solutes. J Am Soc Nephrol 2011; 22:1769-76.
12. Eloot S, Schepers E, Barreto D V, et al. Estimated glomerular filtration rate is a poor predictor of concentration for a broad range of uremic toxins. Clin J Am Soc Nephrol 2011; 6:1266-73.
13. Meyer T W, Hostetter T H. Uremic solutes from colon microbes. Kidney Int 2012; 81:949-54.
14. Mogensen C E. Microalbuminuria, blood pressure and diabetic renal disease: origin and development of ideas. Diabetologia 1999; 42:263-85.
15. Vaidya V S, Niewczas M A, Ficociello L H, et al. Regression of microalbuminuria in type 1 diabetes is associated with lower levels of urinary tubular injury biomarkers, kidney injury molecule-1, and N-acetyl-beta-D-glucosaminidase. Kidney Int 2011; 79:464-70.
16. Cano N J, Fouque D, Leverve X M. Application of branched-chain amino acids in human pathological states: renal failure. J Nutr 2006; 136:299S-307S.
17. Garibotto G, Sofia A, Saffioti S, Bonanni A, Mannucci I, Verzola D. Amino acid and protein metabolism in the human kidney and in patients with chronic kidney disease. Clin Nutr 2010; 29:424-33.
18. Goek O N, Doring A, Gieger C, et al. Serum metabolite concentrations and decreased GFR in the general population. Am J Kidney Dis 2012; 60:197-206.
19. Wang T J, Larson M G, Vasan R S, et al. Metabolite profiles and the risk of developing diabetes. Nat Med 2011; 17:448-53.
20. Zhang H, Saha J, Byun J, et al. Rosiglitazone reduces renal and plasma markers of oxidative injury and reverses urinary metabolite abnormalities in the amelioration of diabetic nephropathy. Am J Physiol Renal Physiol 2008; 295:F1071-F1081.
21. Zhao T, Zhang H, Zhao T, et al. Intrarenal metabolomics reveals the association of local organic toxins with the progression of diabetic kidney disease. J Pharm Biomed Anal 2012; 60:32-43.
22. Makinen V P, Soininen P, Forsblom C, et al. Diagnosing diabetic nephropathy by 1H NMR metabonomics of serum. MAGMA 2006; 19:281-96.
23. Makinen V P, Tynkkynen T, Soininen P, et al. Metabolic diversity of progressive kidney disease in 325 patients with type 1 diabetes (the FinnDiane Study). J Proteome Res 2012; 11:1782-90.
24. van der Kloet F M, Tempels F W, Ismail N, et al. Discovery of early-stage biomarkers for diabetic kidney disease using ms-based metabolomics (FinnDiane study). Metabolomics 2012; 8:109-19.
25. Zhang J, Yan L, Chen W, et al. Metabonomics research of diabetic nephropathy and type 2 diabetes mellitus based on UPLC-oaTOF-MS system. Anal Chim Acta 2009; 650:16-22.
26. Niewczas M A, Gohda T, Skupien J, et al. Circulating TNF Receptors 1 and 2 Predict ESRD in Type 2 Diabetes. J Am Soc Nephrol 2012. J Am Soc Nephrol 23(3):507-515.
27. Floegel A, Drogan D, Wang-Sattler R, et al. Reliability of serum metabolite concentrations over a 4-month period using a targeted metabolomic approach. PLoS One 2011; 6:e21103.
28. Milligan, G. V. A Review Of Monte Carlo Tests Of Cluster Analysis. Multivariate Behavioral Research. 16(3), 379-407, 1981.
29. Xia J, Wishart D S. Web-based inference of biological patterns, functions and pathways from metabolomic data using MetaboAnalyst. Nat Protoc 2011; 6:743-60.
30. Itoh Y, Ezawa A, Kikuchi K, Tsuruta Y, Niwa T. Protein-bound uremic toxins in hemodialysis patients measured by liquid chromatography/tandem mass spectrometry and their effects on endothelial ROS production. Anal Bioanal Chem 2012; 403:1841-50.
31. Meijers B K, Claes K, Bammens B, et al. p-Cresol and cardiovascular risk in mild-to-moderate kidney disease. Clin J Am Soc Nephrol 2010; 5:1182-9.
32. Nicholson J K, Holmes E, Kinross J, et al. Host-gut microbiota metabolic interactions. Science 2012; 336:1262-7.
33. Go A S, Chertow G M, Fan D, McCulloch C E, Hsu C Y. Chronic kidney disease and the risks of death, cardiovascular events, and hospitalization. N Engl J Med 2004; 351:1296-305.
34. Boudonck K J, Mitchell M W, Nemet L, et al. Discovery of metabolomics biomarkers for early detection of nephrotoxicity. Toxicol Pathol 2009; 37:280-92.
35. Clements R S, Jr., DeJesus P V, Jr., Winegrad A I. Raised plasma-myoinositol levels in uraemia and experimental neuropathy. Lancet 1973; 1:1137-41.
36. Amer R J, Prabhu K S, Thompson J T, Hildenbrandt G R, Liken A D, Reddy C C. myo-Inositol oxygenase: molecular cloning and expression of a unique enzyme that oxidizes myo-inositol and D-chiro-inositol. Biochem J 2001; 360:313-20.
37. Nayak B, Kondeti V K, Xie P, et al. Transcriptional and posttranslational modulation of myo-inositol oxygenase by high glucose and related pathobiological stresses. J Biol Chem 2011; 286:27594-611.
38. Berry G T, Mallee J J, Kwon H M, et al. The human osmoregulatory Na+/myo-inositol cotransporter gene (SLC5A3): molecular cloning and localization to chromosome 21. Genomics 1995; 25:507-13.
39. Mallee J J, Atta M G, Lorica V, et al. The structural organization of the human Na+/myo-inositol cotransporter (SLC5A3) gene and characterization of the promoter. Genomics 1997; 46:459-65.
40. Wang S, Mitu G M, Hirschberg R. Osmotic polyuria: an overlooked mechanism in diabetic nephropathy. Nephrol Dial Transplant 2008; 23:2167-72.
41. Kennington A S, Hill C R, Craig J, et al. Low urinary chiro-inositol excretion in non-insulin-dependent diabetes mellitus. N Engl J Med 1990; 323:373-8.
42. Lamer J, Brautigan D L, Thorner M O. D-chiro-inositol glycans in insulin signaling and insulin resistance. Mol Med 2010; 16:543-52.
43. Daughaday W H, Lamer J. The renal excretion of inositol in normal and diabetic human beings. J Clin Invest 1954; 33:326-32.
44. Ziyadeh F N, Simmons D A, Snipes E R, Goldfarb S. Effect of myo-inositol on cell proliferation and collagen transcription and secretion in proximal tubule cells cultured in elevated glucose. J Am Soc Nephrol 1991; 1:1220-9.
45. Bartnicki P, Zbrog Z, Baj Z, Tchorzewski H, Luciak M. Myoinositol may be a factor in uremic immune deficiency. Clin Nephrol 1997; 47:197-201.
46. Charette M, Gray M W. Pseudouridine in RNA: what, where, how, and why. IUBMB Life 2000; 49:341-51.
47. Seidel A, Brunner S, Seidel P, Fritz G I, Herbarth O. Modified nucleosides: an accurate tumour marker for clinical diagnosis of cancer, early detection and therapy control. Br J Cancer 2006; 94:1726-33.
48. Daniewska-Michalska D, Motyl T, Gellert R, et al. Efficiency of hemodialysis of pyrimidine compounds in patients with chronic renal failure. Nephron 1993; 64:193-7.
49. Gerrits G P, Monnens L A, De Abreu R A, Schroder C H, Trijbels J M, Gabreels F J. Disturbances of cerebral purine and pyrimidine metabolism in young children with chronic renal failure. Nephron 1991; 58:310-4.
50. Niwa T, Takeda N, Yoshizumi H. RNA metabolism in uremic patients: accumulation of modified ribonucleosides in uremic serum. Technical note. Kidney Int 1998; 53:1801-6.
51. www.genome.jp/kegg/pathway.html. Accessed Apr. 15, 2013
52. www.hmdb.ca Accessed Apr. 15, 2013
53. Huang S, Mahanta N, Begley T P, Ealick S E. Pseudouridine monophosphate glycosidase: a new glycosidase mechanism. Biochemistry 2012; 51:9245-55.
54. Bernert J T, Jr., Bell C J, Guntupalli J, Hannon W H. Pseudouridine is unsuitable as an endogenous renal clearance marker. Clin Chem 1988; 34:1011-7.
55. Vaziri N D, Freel R W, Hatch M. Effect of chronic experimental renal insufficiency on urate metabolism. J Am Soc Nephrol 1995; 6:1313-7.
56. Anzai N, Kanai Y, Endou H. New insights into renal transport of urate. Curr Opin Rheumatol 2007; 19:151-7.
57. Ficociello L H, Rosolowsky E T, Niewczas M A, et al. High-Normal Serum Uric Acid Increases Risk of Early Declining Renal Function In Type 1 Diabetes: Results of 6-year Follow-up. Diabetes Care 2010; 33(6):1337-1343.
58. Chiang C K, Tanaka T, Nangaku M. Dysregulated oxygen metabolism of the kidney by uremic toxins: review. J Ren Nutr 2012; 22:77-80.
59. Kawakami T, Inagi R, Wada T, Tanaka T, Fujita T, Nangaku M. Indoxyl sulfate inhibits proliferation of human proximal tubular cells via endoplasmic reticulum stress. Am J Physiol Renal Physiol 2010; 299:F568-F576.
60. Ahn S Y, Bhatnagar V. Update on the molecular physiology of organic anion transporters. Curr Opin Nephrol Hypertens 2008; 17:499-505.
61. Wang L, Sweet D H. Renal Organic Anion Transporters (SLC22 Family): Expression, Regulation, Roles in Toxicity, and Impact on Injury and Disease. AAPS J 2012.
62. Monica T A, Mac L M, Muller A, Brandoni A, Anzai N, Endou H. Altered renal elimination of organic anions in rats with chronic renal failure. Biochim Biophys Acta 2005; 1740:29-37.
63. Naud J, Michaud J, Beauchemin S, et al. Effects of chronic renal failure on kidney drug transporters and cytochrome P450 in rats. Drug Metab Dispos 2011; 39:1363-9.
64. Motojima M, Hosokawa A, Yamato H, Muraki T, Yoshioka T. Uremic toxins of organic anions up-regulate PAI-1 expression by induction of NF-kappaB and free radical in proximal tubular cells. Kidney Int 2003; 63:1671-80.
65. Broer S. Amino acid transport across mammalian intestinal and renal epithelia. Physiol Rev 2008; 88:249-86.
66. Williams R E, Major H, Lock E A, Lenz E M, Wilson I D. D-Serine-induced nephrotoxicity: a HPLC-TOF/MS-based metabonomics approach. Toxicology 2005; 207:179-90.
67. Verrey F, Ristic Z, Romeo E, et al. Novel renal amino acid transporters. Annu Rev Physiol 2005; 67:557-72.
68. Verrey F, Singer D, Ramadan T, Vuille-dit-Bille R N, Mariotta L, Camargo S M. Kidney amino acid transport. Pflugers Arch 2009; 458:53-60.
69. Furmanek A, Hofsteenge J. Protein C-mannosylation: facts and questions. Acta Biochim Pol 2000; 47:781-9.
70. Ihara Y, Manabe S, Kanda M, et al. Increased expression of protein C-mannosylation in the aortic vessels of diabetic Zucker rats. Glycobiology 2005; 15:383-92.
71. Yonemura K, Takahira R, Yonekawa O, Wada N, Hishida A. The diagnostic value of serum concentrations of 2-(alpha-mannopyranosyl)-L-tryptophan for normal renal function. Kidney Int 2004; 65:1395-9.
72. Reuter S E, Evans A M. Carnitine and acylcarnitines: pharmacokinetic, pharmacological and clinical aspects. Clin Pharmacokinet 2012; 51:553-72.
73. Fouque D, Holt S, Guebre-Egziabher F, et al. Relationship between serum carnitine, acylcarnitines, and renal function in patients with chronic renal disease. J Ren Nutr 2006; 16:125-31.
74. Sun J, Shannon M, Ando Y, et al. Serum metabolomic profiles from patients with acute kidney injury: a pilot study. J Chromatogr B Analyt Technol Biomed Life Sci 2012; 893-894:107-13.
75. K/DOQI clinical practice guidelines for chronic kidney disease: evaluation, classification, and stratification. Am J Kidney Dis 2002; 39:S1-266.
76. CDC/National Center for Health Statistics.Data Access—National Death Index. Queried on Oct. 10, 2010
77. van den Berg R A, Rubingh C M, Westerhuis J A, van der Werf M J, Smilde A K. Metabolomics data exploration guided by prior knowledge. Anal Chim Acta 2009; 651:173-81.
78. Storey J D, Tibshirani R. Statistical significance for genomewide studies. Proc Natl Acad Sci USA 2003; 100:9440-5.
79. Kugler F, Graneis S, Schreiter P P et al.: Determination of free amino compounds in betalainic fruits and vegetables by gas chromatography with flame ionization and mass spectrometric detection. J Agric Food Chem 54:4311-4318, 2006
80. Rosolowsky E T, Ficociello L H, Maselli N J et al.: High-normal serum uric acid is associated with impaired glomerular filtration rate in nonproteinuric patients with type 1 diabetes. Clin J Am Soc Nephrol 3:706-713, 2008

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of predicting risk of developing renal disease in a patient who has diabetes, comprising:
a) determining the levels of at least three metabolites selected from the group consisting of pseudouridine, C-glycosyltryptophan, myoinositol, threitol, p-cresol sulfate, 2-hydroxyisovalerate, 2-hydroxyisocaproate, glutaryl carnitine, N2, N2-dimethylguanosine, phenylacetylglutamine, arabitol, gulono-1,4-lactone, erythritol, erythronate, N4-acetylcytidine, urate, 2-hydroxyisocaproate, 2-oxoisoleucine, and 2-oxoisocaproate in a plasma or serum sample taken from the patient;
b) comparing the levels of the metabolites in the sample from the patient to control levels of the metabolites; and
c) predicting that the patient is at risk for developing renal disease when the levels of the metabolites in the sample from the patient are significantly higher than the control levels of the metabolites.

2. The method of claim 1, wherein the patient has type 2 diabetes.

3. The method of claim 1, wherein the patient has type 1 diabetes.

4. The method of claim 1, wherein the patient has normal renal function.

5. The method of claim 1, wherein the patient does not have symptoms of renal disease.

6. The method of claim 1, wherein the renal disease is diabetic nephropathy.

7. The method of claim 1, wherein the renal disease is end-stage renal disease (ESRD).

8. The method of claim 1, wherein the sample taken from the patient is a plasma sample.

9. The method of claim 1, wherein the levels of the metabolites are determined using mass spectrometry.

10. A method of identifying a patient who has diabetes as being in need of a therapy to prevent or delay the onset of a renal disease, comprising:
a) determining the levels of at least three metabolites selected from the group consisting of pseudouridine, C-glycosyltryptophan, myoinositol, threitol, p-cresol sulfate, 2-hydroxyisovalerate, 2-hydroxyisocaproate, glutaryl carnitine, N2, N2-dimethylguanosine, phenylacetylglutamine, arabitol, gulono-1,4-lactone, erythritol, erythronate, N4-acetylcytidine, urate, 2-hydroxyisocaproate, 2-oxoisoleucine, and 2-oxoisocaproate in a plasma or serum sample taken from the patient;
b) comparing the levels of the metabolites in the sample from the patient to control levels of the metabolites; and
c) implementing a therapy to prevent or delay the onset of a renal disease in the patient when the levels of the metabolites in the sample from the patient are significantly higher than the control levels of the metabolites.

11. The method of claim 10, wherein the patient has type 2 diabetes.

12. The method of claim 10, wherein the patient has type 1 diabetes.

13. The method of claim 10, wherein the patient has normal renal function.

14. The method of claim 10, wherein the patient does not have symptoms of renal disease.

15. The method of claim 10, wherein the renal disease is diabetic nephropathy.

16. The method of claim 10, wherein the renal disease is end-stage renal disease (ESRD).

17. The method of claim 10, wherein the sample taken from the patient is a plasma sample.

18. The method of claim 10, wherein the levels of the metabolites are determined using mass spectrometry.

19. The method of claim 10, wherein the therapy to prevent or delay the onset of a renal disease comprises administration of drugs to treat hypertension, dietary changes, exercise, weight loss, glycemic control, proteinuria therapies, and albuminuria therapies.
